(19) United States
(12) Patent Application Publication
(10) Pub. No.: US 2023/0272485 A1
(43) Pub. Date: Aug. 31, 2023

(54) REPLICATION STRESS PATHWAY AGENT COMPOSITIONS AND METHODS FOR TREATING CANCER

(71) Applicant: Boundless Bio, Inc., San Diego, CA (US)

(72) Inventors: Christian HASSIG, San Diego, CA (US); Ryan HANSEN, San Diego, CA (US); Snezana MILUTINOVIC, San Diego, CA (US); Zachary D. HORNBY, San Diego, CA (US); Sudhir CHOWDHRY, San Diego, CA (US); Kristen TURNER, San Diego, CA (US); Deepti WILKINSON, San Diego, CA (US); Shailaja KASIBHATLA, San Diego, CA (US)

(21) Appl. No.: 18/169,797

(22) Filed: Feb. 15, 2023

Related U.S. Application Data
(60) Provisional application No. 63/310,968, filed on Feb. 16, 2022.

(51) Int. Cl. C12Q 1/6886; A61K 31/517; A61K 45/06

(52) U.S. Cl. CPC C12Q 1/6886; A61K 31/517; A61K 45/06

(57) ABSTRACT

Provided herein are methods of treating cancer in a subject, wherein the cancer is extrachromosomal DNA-positive (ecDNA-positive) or therapeutically resistant, the method comprising administering to the subject a therapeutically effective amount of a replication stress (RS) pathway agent alone or in combination with a targeted therapeutic.

### RELATED APPLICATIONS

This application claims the benefit of U.S. Provisional Application Ser. No. 63/310,968, filed Feb. 16, 2022, which is hereby incorporated by reference in its entirety.

### BACKGROUND

Cancers often prove resistant to the therapeutics that are used to treat them, frustrating efforts to extend progression free survival in cancer patients. In some cases, treatment resistant cancers are observed to be positive for extrachromosomal DNA (ecDNA), which sometimes contains amplified oncogenes, contributing to therapeutic resistance.

### SUMMARY

In an aspect, there are provided methods for treating a melanoma. In some embodiments, the method comprises identifying a subject suffering from or diagnosed with the melanoma, wherein tumor cells of the melanoma are resistant, are reduced in responsiveness, or non-responsive to a therapeutic treatment; treating the subject with a replication stress pathway agent (RSPA) in an amount sufficient to induce replication stress in the tumor cells; and whereby the growth of the tumor cells, the size of the tumor cells, or the number of the tumor cells is reduced. In some embodiments, the tumor cells comprise BRAF with an amino acid substitution in codon 600 of BRAF. In some embodiments, the tumor cells comprise an amplification of BRAF. In some embodiments, the amplification of BRAF has an amino acid substitution in codon 600 of BRAF. In some embodiments, the tumor cells comprise ecDNA. In some embodiments, the ecDNA comprises nucleic acid encoding BRAF or a portion thereof. In some embodiments, the subject has been previously treated or concurrently treated with a BRAF inhibitor, a MEK inhibitor, or a combination thereof. In some embodiments, the BRAF inhibitor is selected from the group consisting of dabrafenib, emurafenib, encorafenib, KIN-2787, vemurafenib, and an analog thereof. In some embodiments, the MEK inhibitor is selected from the group consisting of binimetinib, cobimetinib, selumetinib, trametinib, and an analog thereof. In some embodiments, the tumor cells comprise homogenously staining region (HSR). In some embodiments, the method further comprises the step of assessing the tumor cells for a presence, an amount, or a change in the presence or the amount of ecDNA. In some embodiments, the step of assessing comprises one or more of FISH, whole genome sequencing, whole exome sequencing, targeted panel sequencing, assaying for one or more biomarkers of ecDNA, and assaying for a presence or an amount of an oncogene amplification. In some embodiments, the RSPA is selected from the group consisting of a RNR inhibitor, an ATR inhibitor, a CHK1 inhibitor, a WEE1 inhibitor, and a PARG inhibitor. In some embodiments, the RNR inhibitor is selected from the group consisting of 5-chloro-2-(n-((1S,2R)-2-(6-fluoro-2,3-dimethylphenyl)-1-(5-oxo-4,5-dihydro-1,3,4-oxadiazol-2-yl)propyl)sulfamoyl)benzamide, cladribine, clofarabine, COH29 (N-[4-(3,4-dihydroxyphenyl)-5-phenyl-1,3-thiazol-2-yl]-3,4-dihydroxybenzamide), fludarabine, gemcitabine, hydroxyurea, motexafin gadolinium, tezacitabine, and triapine. In some embodiments, the ATR inhibitor is selected from the group consisting of ART-0380, ATRN-119, ATRN-212, AZ-20, AZZ-6738, BAY-1895344, berzosertib (M-6620, VX-970; VE-822), BKT-300, IMP-9064, M-1774, M-4344 (VX-803), M-6620, nLs-BG-129, NU-6027, RP-3500, and SC-0245. In some embodiments, the CHK1 inhibitor is selected from the group consisting of AZD-7762, BEBT-260, GDC-0575, LY-2880070, PF-477736, prexasertib (ACR-368), rabusertib (LY-2603618), RG-7602, SCH-900776, SRA737, and XCCS-605B. In some embodiments, the WEE1 inhibitor is selected from the group consisting of AZD1775 (MK1775), Bos-I, bosutinib, DC-859/A, Debio 0123, IMP7068, NUV-569, PD0166285, PD0407824, SC-0191, SDR-7778, SDR-7995, and ZN-c3. In some embodiments, the PARG inhibitor is selected from the group consisting of PD00017273, 3-((1-Methyl-1H-pyrazol-4-yl)methyl)-N-(1-methylcyclopropyl)-1-(oxetan-3-ylmethyl)-2,4-dioxo-1,2,3,4-tetrahydroquinazoline-6-sulfonamide, and (R)—N-(1-Cyanocyclopropyl)-1-(5-(difluoromethyl)-1,3,4-thiadiazol-2-yl)-4-(3-methyl-4-(1-methylcyclopropane-1-carbonyl)piperazin-1-yl)-1H-indazole-6-sulfonamide.

In another aspect, there are provided methods for treating a neuroblastoma. In some embodiments, the method comprises identifying a subject suffering from or diagnosed with the neuroblastoma, wherein tumor cells of the neuroblastoma are resistant, are reduced in responsiveness, or non-responsive to a therapeutic treatment; treating the subject with a replication stress pathway agent (RSPA) in an amount sufficient to induce replication stress in the tumor cells; and whereby the growth of the tumor cells, the size of the tumor cells, or the number of the tumor cells is reduced. In some embodiments, the tumor cells comprise an amplification of CDK4, MDM2, MYCN, or any combination thereof. In some embodiments, the tumor cells are upregulated for one or more of a drug efflux pump or a cancer stem cell marker. In some embodiments, the one or more of the drug efflux pump or the cancer stem cell marker comprise ABCB1, ABCC1-ABCC6, ABCC10-ABCC12, ABCG2, BCL-2L1, CD24, CD44, MCL-1, GL1, NOTCH-1, NOTCH-2, NOTCH-3, and SOX2. In some embodiments, the tumor cells comprise ecDNA. In some embodiments, the ecDNA comprises nucleic acid encoding CDK4, MDM2, MYCN, or a portion thereof. In some embodiments, the subject has been previously treated or concurrently treated with a standard of care (SOC) therapeutic agent. In some embodiments, the SOC is selected from the group consisting of cisplatin, carboplatin, cyclophosphamide, doxorubicin, etoposide, vincristine, and an analog thereof. In some embodiments, the subject has been previously treated or concurrently treated with a therapeutic agent targeted against CDK4, MDM2, or MYCN. In some embodiments, the tumor cells comprise HSR. In some embodiments, the method further comprises the step of assessing the tumor cells for a presence, an amount, or a change in the presence or the amount of ecDNA. In some embodiments, the step of assessing comprises one or more of FISH, whole genome sequencing, whole exome sequencing, targeted panel sequencing, assaying for one or more biomarkers of ecDNA, and assaying for a presence or an amount of an oncogene amplification. In some embodiments, the RSPA is selected from the group consisting of a RNR inhibitor, an ATR inhibitor, a CHK1 inhibitor, a WEE1 inhibitor, and a PARG inhibitor. In some embodiments, the RNR inhibitor is selected from the group consisting of 5-chloro-2-(n-((1S,2R)-2-(6-fluoro-2,3-dimethylphenyl)-1-(5-oxo-4,5-dihydro-1,3,4-oxadiazol-2-yl)propyl)sulfamoyl)benzamide, cladribine, clofarabine, COH29 (N-[4-(3,4-dihydroxyphenyl)-5-phenyl-1,3-thiazol-2-yl]-3,4-dihydroxybenzamide), fludarabine, gemcitabine, hydroxyurea, motexafin gadolinium, tezacitabine, and triapine. In some embodiments, the ATR inhibitor is selected from the group consisting of ART-0380, ATRN-119, ATRN-212, AZ-20, AZZ-6738, BAY-1895344, berzosertib (M-6620, VX-920; VE-822), BKT-300, IMP-9064, M-1774, M-4344 (VX-803), M-6620, nLs-BG-129, NU-6027, RP-3500, and SC-0245. In some embodiments, the CHK1 inhibitor is selected from the group consisting of AZD-7762, BEBT-260, GDC-0575, LY-2880070, PF-477736, prexasertib (ACR-368), rabusertib (LY-2603618), RG-7602, SCH-900776, SRA737, and XCCS-605B. In some embodiments, the WEE1 inhibitor is selected from the group consisting of AZD1775 (MK1775), Bos-I, bosutinib, DC-8591A, Debio 0123, IMP7068, NUV-569, PD0166285, PD0407824, SC-0191, SDR-7778, SDR-7995, and ZN-c3. In some embodiments, the PARG inhibitor is selected from the group consisting of PD00017273, 3-((1-Methyl-1H-pyrazol-4-yl)methyl)-N-(1-methylcyclopropyl)-1-(oxetan-3-ylmethyl)-2,4-dioxo-1,2,3,4-tetrahydroquinazoline-6-sulfonamide, and (R)—N-(1-Cyanocyclopropyl)-1-(5-(difluoromethyl)-1,3,4-thiadiazol-2-yl)-4-(3-methyl-4-(1-methylcyclopropane-1-carbonyl)piperazin-1-yl)-1H-indazole-6-sulfonamide.

In another aspect, there are provided methods for treating a gastric cancer. In some embodiments, the method comprises identifying a subject suffering from or diagnosed with the gastric cancer, wherein tumor cells of the gastric cancer are resistant, are reduced in responsiveness, or non-responsive to a therapeutic treatment; treating the subject with a replication stress pathway agent (RSPA) in an amount sufficient to induce replication stress in the tumor cells; and whereby the growth of the tumor cells, the size of the tumor cells, or the number of the tumor cells is reduced. In some embodiments, the tumor cells comprise an amplification of FGFR2. In some embodiments, the tumor cells comprise ecDNA. In some embodiments, the ecDNA comprises nucleic acid encoding FGFR2, or a portion thereof. In some embodiments, the subject has been previously treated or concurrently treated with an FGFR2 inhibitor. In some embodiments, the FGFR2 inhibitor is selected from the group consisting of erdafitinib, infigratinib, KIN-3248, pemigatinib, RLY-4008, TYRA-200, and an analog thereof. In some embodiments, the tumor cells comprise HSR. In some embodiments, the method further comprises the step of assessing the tumor cells for a presence, an amount, or a change in the presence or the amount of ecDNA. In some embodiments, the step of assessing comprises one or more of FISH, whole genome sequencing, whole exome sequencing, targeted panel sequencing, assaying for one or more biomarkers of ecDNA, and assaying for a presence or an amount of an oncogene amplification. In some embodiments, the RSPA is selected from the group consisting of a RNR inhibitor, an ATR inhibitor, a CHK1 inhibitor, a WEE1 inhibitor, and a PARG inhibitor. In some embodiments, the RNR inhibitor is selected from the group consisting of 5-chloro-2-(n-((1S,2R)-2-(6-fluoro-2,3-dimethylphenyl)-1-(5-oxo-4,5-dihydro-1,3,4-oxadiazol-2-yl)propyl)sulfamoyl)benzamide, cladribine, clofarabine, COH29 (N-[4-(3,4-dihydroxyphenyl)-5-phenyl-1,3-thiazol-2-yl]-3,4-dihydroxybenzamide), fludarabine, gemcitabine, hydroxyurea, motexafin gadolinium, tezacitabine, and triapine. In some embodiments, the ATR inhibitor is selected from the group consisting of ART-0380, ATRN-119, ATRN-212, AZ-20, AZZ-6738, BAY-1895344, berzosertib (M-6620, VX-970; VE-822), BKT-300, IMP-9064, M-1774, M-4344 (VX-803), M-6620, nLs-BG-129, NU-6027, RP-3500, and SC-0245. In some embodiments, the CHK1 inhibitor is selected from the group consisting of AZD-7762, BEBT-260, GDC-0575, LY-2880070, PF-477736, prexasertib (ACR-368), rabusertib (LY-2603618), RG-7602, SCH-900776, SRA737, and XCCS-605B. In some embodiments, the WEE1 inhibitor is selected from the group consisting of AZD1775 (MK1775), Bos-I, bosutinib, DC-859/A, Debio 0123, IMP7068, NUV-569, PD0166285, PD0407824, SC-0191, SDR-7778, SDR-7995, and ZN-c3. In some embodiments, the PARG inhibitor is selected from the group consisting of PD00017273, 3-((1-Methyl-1H-pyrazol-4-yl)methyl)-N-(1-methylcyclopropyl)-1-(oxetan-3-ylmethyl)-2,4-dioxo-1,2,3,4-tetrahydroquinazoline-6-sulfonamide, and (R)—N-(1-Cyanocyclopropyl)-1-(5-(difluoromethyl)-1,3,4-thiadiazol-2-yl)-4-(3-methyl-4-(1-methylcyclopropane-1-carbonyl)piperazin-1-yl)-1H-indazole-6-sulfonamide.

In another aspect, there are provided methods for treating an esophageal cancer. In some embodiments, the method comprises identifying a subject suffering from or diagnosed with the esophageal cancer, wherein tumor cells of the esophageal cancer are resistant, are reduced in responsiveness, or non-responsive to a therapeutic treatment; treating the subject with a replication stress pathway agent (RSPA) in an amount sufficient to induce replication stress in the tumor cells; and whereby the growth of the tumor cells, the size of the tumor cells, or the number of the tumor cells is reduced. In some embodiments, the tumor cells comprise an amplification of EGFR or MDM2. In some embodiments, the tumor cells comprise ecDNA. In some embodiments, the ecDNA comprises nucleic acid encoding EGFR, MDM2, or a portion thereof. In some embodiments, the subject has been previously treated or concurrently treated with an EGFR inhibitor or an MDM2 inhibitor. In some embodiments, EGFR inhibitor is selected from the group consisting of erlotinib, gefitinib, osimertinib, and an analog thereof. In some embodiments, EGFR inhibitor is a biologic that binds to and/or inhibits EGFR selected from the group consisting of an antibody, antibody drug conjugate and antigen binding fragment. In some embodiments, antibody for EGFR is selected from cetuximab, panitumumab, nimotuzumab, and necitumumab. In some embodiments, the MDM2 inhibitor is selected from the group consisting of AD-021.32, ALRN-6924, AM-8533, AMG232, ASTX-295, B1907828, HDM201, KT-253, RG-738, MI-43, Milademetan (RAIN32), serdemetan, SIL-43, and PXN527. In some embodiments, the tumor cells comprise HSR. In some embodiments, the method further comprises the step of assessing the tumor cells for a presence, an amount, or a change in the presence or the amount of ecDNA. In some embodiments, the step of assessing comprises one or more of FISH, whole genome sequencing, whole exome sequencing, targeted panel sequencing, assaying for one or more biomarkers of ecDNA, and assaying for a presence or an amount of an oncogene amplification. In some embodiments, the RSPA is selected from the group consisting of a RNR inhibitor, an ATR inhibitor, a CHK1 inhibitor, a WEE1 inhibitor, and a PARG inhibitor. In some embodiments, the RNR inhibitor is selected from the group consisting of 5-chloro-2-(n-((1S,2R)-2-(6-fluoro-2,3-dimethylphenyl)-1-(5-oxo-4,5-dihydro-1,3,4-oxadiazol-2-yl)propyl)sulfamoyl)benzamide, cladribine, clofarabine, COH29 (N-[4-(3,4-dihydroxyphenyl)-5-phenyl-1,3-thiazol-2-yl]-3,4-dihydroxybenzamide), fludarabine, gemcitabine, hydroxyurea, motexafin gadolinium, tezacitabine, and triapine. In some embodiments, the ATR inhibitor is selected from the group consisting of ART-0380, ATRN-119, ATRN-212, AZ-20, AZZ-6738, BAY-1895344, berzosertib (M-6620, VX-970; VE-822), BKT-300, IMP-9064, M-1774, M-4344 (VX-803), M-6620, nLs-BG-129, NU-6027, RP-3500, and SC-0245. In some embodiments, the CHK1 inhibitor is selected from the group consisting of AZD-7762, BEBT-260, GDC-0575, LY-2880070, PF-477736, prexasertib (ACR-368), rabusertib (LY-2603618), RG-7602, SCH-900776, SRA737, and XCCS-605B. In some embodiments, the WEE1 inhibitor is selected from the group consisting of AZD1775 (MK1775), Bos-I, bosutinib, DC-859/A, Debio 0123, IMP7068, NUV-569, PD0166285, PD0407824, SC-0191, SDR-7778, SDR-7995, and ZN-c3. In some embodiments, the PARG inhibitor is selected from the group consisting of PD00017273, 3-((1-Methyl-1H-pyrazol-4-yl)methyl)-N-(1-methylcyclopropyl)-1-(oxetan-3-ylmethyl)-2,4-dioxo-1,2,3,4-tetrahydroquinazoline-6-sulfonamide, and (R)—N-(1-Cyanocyclopropyl)-1-(5-(difluoromethyl)-1,3,4-thiadiazol-2-yl)-4-(3-methyl-4-(1-methylcyclopropane-1-carbonyl)piperazin-1-yl)-1H-indazole-6-sulfonamide.

In another aspect, there are provided methods for treating glioblastoma. In some embodiments, the method comprises identifying a subject suffering from or diagnosed with the glioblastoma, wherein tumor cells of the glioblastoma are resistant, are reduced in responsiveness, or non-responsive to a therapeutic treatment; treating the subject with a replication stress pathway agent (RSPA) in an amount sufficient to induce replication stress in the tumor cells; and whereby the growth of the tumor cells, the size of the tumor cells, or the number of the tumor cells is reduced. In some embodiments, the tumor cells comprise an amplification of CDK4, CDK8, and EGFR. In some embodiments, the tumor cells comprise ecDNA. In some embodiments, the ecDNA comprises nucleic acid encoding EGFR, or a portion thereof. In some embodiments, the subject has been previously treated or concurrently treated with an EGFR inhibitor. In some embodiments, the EGFR inhibitor is selected from the group consisting of erlotinib, gefitinib, osimertinib, and an analog thereof. In some embodiments, EGFR inhibitor is a biologic that binds to and/or inhibits EGFR selected from the group consisting of an antibody, antibody drug conjugate and antigen binding fragment. In some embodiments, antibody for EGFR is selected from cetuximab, panitumumab, nimotuzumab, and necitumumab. In some embodiments, the tumor cells comprise HSR. In some embodiments, the method further comprises the step of assessing the tumor cells for a presence, an amount, or a change in the presence or the amount of ecDNA. In some embodiments, the step of assessing comprises one or more of FISH, whole genome sequencing, whole exome sequencing, targeted panel sequencing, assaying for one or more biomarkers of ecDNA, and assaying for a presence or an amount of an oncogene amplification. In some embodiments, the RSPA is selected from the group consisting of a RNR inhibitor, an ATR inhibitor, a CHK1 inhibitor, a WEE1 inhibitor, and a PARG inhibitor. In some embodiments, the RNR inhibitor is selected from the group consisting of 5-chloro-2-(n-((1S,2R)-2-(6-fluoro-2,3-dimethylphenyl)-1-(5-oxo-4,5-dihydro-1,3,4-oxadiazol-2-yl)propyl)sulfamoyl)benzamide, cladribine, clofarabine, COH29 (N-[4-(3,4-dihydroxyphenyl)-5-phenyl-1,3-thiazol-2-yl]-3,4-dihydroxybenzamide), fludarabine, gemcitabine, hydroxyurea, motexafin gadolinium, tezacitabine, and triapine. In some embodiments, the ATR inhibitor is selected from the group consisting of ART-0380, ATRN-119, ATRN-212, AZ-20, AZZ-6738, BAY-1895344, berzosertib (M-6620, VX-970; VE-822), BKT-300, IMP-9064, M-1774, M-4344 (VX-803), M-6620, nLs-BG-129, NU-6027, RP-3500, and SC-0245. In some embodiments, the CHK1 inhibitor is selected from the group consisting of AZD-7762, BEBT-260, GDC-0575, LY-2880070, PF-477736, prexasertib (ACR-368), rabusertib (LY-2603618), RG-7602, SCH-900776, SRA737, and XCCS-605B. In some embodiments, the WEE1 inhibitor is selected from the group consisting of AZD1775 (MK1775), Bos-I, bosutinib, DC-859/A, Debio 0123, IMP7068, NUV-569, PD0166285, PD0407824, SC-0191, SDR-7778, SDR-7995, and ZN-c3. In some embodiments, the PARG inhibitor is selected from the group consisting of PD00017273, 3-((1-Methyl-1H-pyrazol-4-yl)methyl)-N-(1-methylcyclopropyl)-1-(oxetan-3-ylmethyl)-2,4-dioxo-1,2,3,4-tetrahydroquinazoline-6-sulfonamide, and (R)—N-(1-Cyanocyclopropyl)-1-(5-(difluoromethyl)-1,3,4-thiadiazol-2-yl)-4-(3-methyl-4(1-methylcyclopropane-1-carbonyl)piperazin-1-yl)-1H-indazole-6-sulfonamide.

In another aspect, there are provided methods for treating a lung cancer. In some embodiments, the method comprises identifying a subject suffering from or diagnosed with the lung cancer, wherein tumor cells of the lung cancer are resistant, are reduced in responsiveness, or non-responsive to a therapeutic treatment; treating the subject with a replication stress pathway agent (RSPA) in an amount sufficient to induce replication stress in the tumor cells; and whereby the growth of the tumor cells, the size of the tumor cells, or the number of the tumor cells is reduced. In some embodiments, the tumor cells comprise an amplification of EGFR, KRAS, or MYC. In some embodiments, the tumor cells comprise ecDNA. In some embodiments, the ecDNA comprises nucleic acid encoding EGFR, KRAS, MYC, or a portion thereof. In some embodiments, the subject has been previously treated or concurrently treated with an EGFR inhibitor, a KRAS inhibitor, or a MYC inhibitor. In some embodiments, the EGFR inhibitor is selected from the group consisting of erlotinib, gefitinib, osimertinib, and an analog thereof. In some embodiments, EGFR inhibitor is a biologic that binds to and/or inhibits EGFR selected from the group consisting of an antibody, antibody drug conjugate and antigen binding fragment. In some embodiments, antibody for EGFR is selected from cetuximab, panitumumab, nimotuzumab, and necitumumab. In some embodiments, the KRAS inhibitor is selected from the group consisting of adagrasib, sotorasib, and an analog thereof. In some embodiments, the KRAS inhibitor is selected from the group consisting of BT 1823911, D-1553, ERAS-3490, GDC-6036, JDQ443, JNJ-74699157 (ARS-3248), LY3499446, LY3537982, and RM-018. In some embodiments, the subject has been previously treated or concurrently treated with chemotherapy, optionally one or more of paclitaxel, doxorubicin, and vincristine. In some embodiments, the tumor cells comprise a G12C mutation in KRAS. In some embodiments, the tumor cells comprise HSR. In some embodiments, the method further comprises the step of assessing the tumor cells for a presence, an amount, or change in the presence or the amount of ecDNA. In some embodiments, the step of assessing comprises one or more of FISH, whole genome sequencing, whole exome sequencing, targeted panel sequencing, assaying for one or more biomarkers of ecDNA, and assaying for a presence or an amount of an oncogene amplification. In some embodiments, the RSPA is selected from the group consisting of a RNR inhibitor, an ATR inhibitor, a CHK1 inhibitor, a WEE1 inhibitor, and a PARG inhibitor. In some embodiments, the RNR inhibitor is selected from the group consisting of 5-chloro-2-(n-((1S,2R)-2-(6-fluoro-2,3-dimethylphenyl)-1-(5-oxo-4,5-dihydro-1,3,4-oxadiazol-2-yl)propyl)sulfamoyl)benzamide, cladribine, clofarabine, COH29 (N-[4-(3,4-dihydroxyphenyl)-5-phenyl-1,3-thiazol-2-yl]-3,4-dihydroxybenzamide), fludarabine, gemcitabine, hydroxyurea, motexafin gadolinium, tezacitabine, and triapine. In some embodiments, the ATR inhibitor is selected from the group consisting of ART-0380, ATRN-119, ATRN-212, AZ-20, AZZ-6738, BAY-1895344, berzosertib (M-6620, VX-920; VE-822), BKT-300, IMP-9064, M-1774, M-4344 (VX-803), M-6620, nLs-BG-129, NU-6027, RP-3500, and SC-0245. In some embodiments, the CHK1 inhibitor is selected from the group consisting of AZD-7762, BEBT-260, GDC-0575, LY-2880070, PF-477736, prexasertib (ACR-368), rabusertib (LY-2603618), RG-7602, SCH-900776, SRA737, and XCCS-605B. In some embodiments, the WEE1 inhibitor is selected from the group consisting of AZD1775 (MK1775), Bos-I, bosutinib, DC-8591A, Debio 0123, IMP7068, NUV-569, PD0166285, PD0407824, SC-0191, SDR-7778, SDR-7995, and ZN-c3. In some embodiments, the PARG inhibitor is selected from the group consisting of PD00017273, 3-((1-Methyl-1H-pyrazol-4-yl)methyl)-N-(1-ethylcyclopropyl)-1-(oxetan-3-ylmethyl)-2,4-dioxo-1,2,3,4-tetrahydroquinazoline-6-sulfonamide, and (R)—N-(1-Cyanocyclopropyl)-1-(5-(difluoromethyl)-1,3,4-thiadiazol-2-yl)-4-(3-methyl-4-(1-ethylcyclopropane-1-carbonyl)piperazin-1-yl)-1H-indazole-6-sulfonamide.

In another aspect, there are provided methods for identifying a subject responsive to a replication stress pathway agent (RSPA). In some embodiments, the method comprises preparing a first sample and a second sample from a tumor or tumor cell acquired from the subject; wherein the first sample and the second sample are derived from different time points; assessing the first sample and the second sample for a presence and/or a level of ecDNA; comparing the presence and/or the level of ecDNA between the first sample and the second sample; and treating the subject with the RSPA based on a differential the presence and/or the level of ecDNA of the second sample as compared to the first sample. In some embodiments, the first sample is acquired prior to treatment with a first therapeutic agent and the second sample is acquired during the course of treatment or subsequent to treatment with the first therapeutic agent. In some embodiments, the subject is treated with the RSPA if the level of ecDNA is increased in the second sample as compared to the first sample. In some embodiments, the second sample comprises an alteration in the structure of the ecDNA as compared to the first sample. In some embodiments, the alteration comprises an amplification of a gene and/or an oncogene on ecDNA of the second sample as compared to the first sample. In some embodiments, the tumor or tumor cell is a melanoma and wherein the tumor or tumor cells of the second sample are reduced in responsiveness to a mitogen-activated protein kinase (MAPK) inhibitor. In some embodiments, the tumor or tumor cell is a melanoma and the oncogene is BRAF. In some embodiments, the BRAF has an amino acid substitution in codon 600 of BRAF. In some embodiments, the subject has been previously treated or concurrently treated with a BRAF inhibitor, a MEK inhibitor, or a combination thereof. In some embodiments, the BRAF inhibitor is selected from the group consisting of dabrafenib, emurafenib, encorafenib, KIN-2787, vemurafenib, and an analog thereof. In some embodiments, the MEK inhibitor is selected from the group consisting of binimetinib, cobimetinib, selumetinib, trametinib, and an analog thereof. In some embodiments, the tumor or tumor cell is a neuroblastoma and wherein the tumor or tumor cells of the second sample are reduced in responsiveness to a standard of care treatment. In some embodiments, the tumor or tumor cell is a neuroblastoma and the oncogene is CDK4, MDM2, MYCN, or a portion thereof. In some embodiments, the subject has been previously treated or concurrently treated with a standard of care (SOC) therapeutic agent selected from the group consisting of cisplatin, carboplatin, cyclophosphamide, doxorubicin, etoposide, vincristine, and an analog thereof. In some embodiments, the subject has been previously treated or concurrently treated with a therapeutic agent targeted against CDK4, MDM2, or MYCN. In some embodiments, the tumor or tumor cell is a gastric cancer and wherein the tumor or tumor cells of the second sample are reduced in responsiveness to a therapeutic agent directed against FGFR2. In some embodiments, the tumor or tumor cell is a gastric cancer and the oncogene is FGFR2. In some embodiments, the subject has been previously treated or concurrently treated with an FGFR2 inhibitor. In some embodiments, the FGFR2 inhibitor is selected from the group consisting of erdafitinib, infigratinib, KIN-3248, erdafitinib, pemigatinib, RLY-4008, TYRA-200, and an analog thereof. In some embodiments, the tumor or tumor cell is an esophageal cancer and wherein the tumor or tumor cells of the second sample are reduced in responsiveness to a therapeutic agent directed against EGFR or MDM2. In some embodiments, the tumor or tumor cell is an esophageal cancer and the oncogene is EGFR or MDM2. In some embodiments, the subject has been previously treated or concurrently treated with an EGFR inhibitor or an MDM2 inhibitor. In some embodiments, the EGFR inhibitor is selected from the group consisting of erlotinib, gefitinib, osimertinib, and an analog thereof. In some embodiments, EGFR inhibitor is a biologic that binds to and/or inhibits EGFR selected from the group consisting of an antibody, antibody drug conjugate and antigen binding fragment. In some embodiments, the MDM2 inhibitor is selected from the group consisting of AD-021.32, ALRN-6924, AM-8533, AMG232, ASTX-295, BI907828, HDM201, KT-253, RG-738, MI-43, Milademetan (RAIN32), serdemetan, SIL-43, and PXN527. In some embodiments, the tumor or tumor cell is a glioblastoma and wherein the tumor or tumor cells of the second sample are reduced in responsiveness to a therapeutic agent directed against CDK4, CDK6, or EGFR. In some embodiments, the tumor or tumor cell is a glioblastoma and the oncogene is EGFR. In some embodiments, the subject has been previously treated or concurrently treated with an EGFR inhibitor. In some embodiments, the EFGR inhibitor is selected from the group consisting of erlotinib, gefitinib, osimertinib, and an analog thereof. In some embodiments, the tumor or tumor cell is a lung cancer and wherein the tumor or tumor cells of the second sample are reduced in responsiveness to a therapeutic agent directed against EGFR, KRAS, or MYC. In some embodiments, the tumor or tumor cell is a lung cancer and the oncogene is EGFR, KRAS, or MYC. In some embodiments, the subject has been previously treated or concurrently treated with an EGFR inhibitor, a KRAS inhibitor, or a MYC inhibitor. In some embodiments, the EFGR inhibitor is selected from the group consisting of erlotinib, gefitinib, osimertinib, and an analog thereof. In some embodiments, the KRAS inhibitor is selected from the group consisting of adagrasib, sotorasib, and an analog thereof. In some embodiments, the KRAS inhibitor is selected from the group consisting of BI 1823911, D-1553, ERAS-3490, GDC-6036, JDQ443, JNJ-74699157 (ARS-3248), LY3499446, LY3537982, and RM-018. In some embodiments, the subject has been previously treated or concurrently treated with chemotherapy, optionally one or more of paclitaxel, doxorubicin, and vincristine. In some embodiments, the subject harbors a G12C mutation in KRAS. In some embodiments, the step of assessing comprises one or more of FISH, whole genome sequencing, whole exome sequencing, targeted panel sequencing, assaying for one or more biomarkers of ecDNA, and assaying for a presence or an amount of an oncogene amplification. In some embodiments, the RSPA is selected from the group consisting of a RNR inhibitor, an ATR inhibitor, a CHK1 inhibitor, a WEE1 inhibitor, and a PARG inhibitor. In some embodiments, the structure of ecDNA is assessed with Amplicon Architect.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present invention will be obtained by reference to the following detailed description that sets forth illustrative embodiments, in which the principles of the invention are utilized, and the accompanying drawings of which:

FIG. 1 shows amplification of MYC on ecDNA in neuroblastoma cell lines (left: CHP-126; center: CHP-212; right: MCIXC).

FIG. 2A shows DAPI staining along with the identified MYC amplified and FGFR2-amplified ecDNA.

FIG. 2B shows that change of the copy number of MYC, FGFR2, and EGFR in a gastric cancer cell line after treatment with FGFR inhibitor.

FIG. 2C shows amplification of EGFR on ecDNA in a gastric cancer cell line.

FIG. 3A shows inhibition of tumor growth of gastric cancer in mice by ecDTx CHK1 inhibitor, infigratinib, and a combination thereof.

FIG. 3B shows inhibition of copy number of MYC and FGFR2 in mice by ecDTx CHK1 inhibitor, infigratinib, and the combination thereof.

FIG. 3C shows the presence of FRGF2 and MYC amplification on ecDNA.

FIG. 4A shows inhibition of tumor growth of gastric cancer in mice by ecDTx CHK1 inhibitor, infigratinib, and the combination thereof, in a 7-day multiple dosing experiment.

FIG. 4B shows that the combination of ecDTx CHK1 inhibitor with infigratinib significantly decreases tumor volume as compared to the vehicle control and the single agents.

FIG. 4C shows that the copy number of MYC is similar in the treatment groups but the copy number of FGFR2 in the infigratinib alone is dramatically higher than the other groups.

FIG. 4D shows MYC and FGFR2 expression in the gastric tumors of the treated mice.

FIG. 5A shows amplification of MDM2 on ecDNA in a sarcoma PDX model.

FIG. 5B shows inhibition of tumor growth of sarcoma in mice by vehicle only, ecDTx only, a targeted agent only, and a combination of the targeted agent+ecDTx.

FIG. 6A shows a significant increase in MYC amplification on ecDNA in cells treated with the EGFR inhibitor erlotinib as compared to control cells.

FIG. 6B shows amplification of MYC on ecDNA in the control cells and the erlotinib-treated cells.

FIG. 7A shows amplification of ABCB1 transporter on ecDNA in a lung cancer cell line.

FIG. 7B shows the copy number of ABCB1 transporter amplified ecDNA in control cells and paclitaxel-treated cells.

FIG. 7C shows a significant increase in the gene copy number of ABCB1 transporter in the paclitaxel-treated cells as compared to the control cells.

FIG. 8A shows the presence of paclitaxel resistant cells in a colorectal cancer cell line.

FIG. 8B shows a high copy number of ecDNA harboring the ABCB1 drug efflux pump in the paclitaxel resistant cells.

FIG. 8C shows amplification of various genes in the paclitaxel resistant cells.

FIG. 9 shows the presence of ecDNA+ tumor cells in a variety of cancer types.

FIG. 10A shows inhibition of tumor proliferation of lung carcinoma in ecDNA+ cells.

FIG. 10B shows inhibition of tumor proliferation of colon adenocarcinoma in ecDNA+ cells.

FIG. 10C shows inhibition of tumor proliferation of gastric carcinoma, astrocytoma, pancreatic adenocarcinoma, and neuroblastoma in ecDNA+ cells.

FIG. 11 shows a comparison of sensitivity to CHK1 inhibition in cell lines with and without amplified oncogenes.

### DETAILED DESCRIPTION

Numerous oncogene-directed therapies have demonstrated clinical efficacy against mutated or activated fusion oncogene targets, however these same therapies do not always yield good objective response rate (ORR) or progression-free survival (PFS) against tumors, especially when the same oncogene is amplified. Despite considerable effort, the oncology field has failed to address this significant unmet need cancer population characterized by amplified oncogenes. Data suggests a substantial proportion of these amplifications are focal amplifications that in some cases occur on extrachromosomal DNA (ecDNA), and this ecDNA phenomenon can account for lack of treatment success.

The oncology field has struggled to find the appropriate genetic background/sensitivity signature to successfully deploy Replication Stress (RS)-targeted therapies including ATR, CHK1 and WEE1. ATR inhibitors are showing some potential in ATM-mutant prostate cancer, but studies are ongoing. Synthetic lethality associated with oncogene amplification has been proposed (such as MYC, MYCN, MYCL, CCNE1 in particular, as they have been associated with increased RS), along with other genetic alterations and/or HPV+. The data supporting these dependencies were far from conclusive and too heterogeneous.

Provided herein are methods for treating cancer with a replication stress pathway agent (RSPA). In some cases, the cancer comprises melanoma, neuroblastoma, gastric cancer, esophageal cancer, glioblastoma, and lung cancer. In some cases, a subject is identified as suffering from or diagnosed with a cancer. In some cases, the tumor cells of the cancer are resistant, are reduced in responsiveness, or non-responsive to a therapeutic treatment. In some cases, the subject is treated with a replication stress pathway agent (RSPA) in an amount sufficient to induce replication stress in the tumor cells. In some cases, the treatment with a replication stress pathway agent (RSPA) results in reduced growth of the tumor cells, the size of the tumor cells, or the number of the tumor cells in the subject.

Also provided herein are methods for identifying a subject responsive to a replication stress pathway agent (RSPA). In some cases, a first sample and a second sample are obtained from a tumor or tumor cell acquired from the subject. In some cases, the first and second samples are derived from different time points. In some cases, a presence and/or a level of ecDNA are assessed in the first sample and the second sample. In some cases, the presence and/or the level of ecDNA of the second sample are compared to the first sample. In some cases, the subject is treated with RSPA based on a differential presence and/or level of ecDNA of the second sample as compared to the first sample.

### Methods of Treatment

Melanoma

In an aspect, provided herein are methods of treating cancer in a subject, for example methods of treating a melanoma in a subject. In some cases, methods herein comprise treating the subject with a replication stress pathway agent (RSPA). In some cases, the replication stress pathway agent (RSPA) is used in an amount sufficient to induce replication stress in the tumor cells. In some cases, the method further comprises identifying a subject suffering from or diagnosed with a melanoma. In some cases, tumor cells of the melanoma in the subject are resistant, are reduced in responsiveness, or non-responsive to a therapeutic treatment. In some cases, the treatment with a replication stress pathway agent (RSPA) reduces the growth of the tumor cells, the size of the tumor cells, or the number of the tumor cells.

In an aspect of methods herein, the tumor cells comprise BRAF or an amplification of BRAF. In some cases, the BRAF has an amino acid substitution. In some cases, the the tumor cells comprise BRAF or an amplification of BRAF where the BRAF has an amino acid substitution in codon 600 of BRAF.

In an aspect of methods provided herein, the tumor cells comprise ecDNA. In some cases, the ecDNA comprises nucleic acid encoding BRAF, a portion thereof, or an amplification thereof. In some cases, the ecDNA comprises an amplification of BRAF where the nucleic acid encoding BRAF or a portion thereof encodes an amino acid substitution in codon 600 of BRAF.

In an aspect of methods provided herein, the subject has been previously treated or concurrently treated with a therapeutic agent. In some cases, the subject has been previously treated or concurrently treated with a BRAF inhibitor, a MEK inhibitor, or a combination thereof. In some cases, the BRAF inhibitor is selected from the group consisting of dabrafenib, emurafenib, encorafenib, KIN-2787, vemurafenib, and an analog thereof. In some cases, the MEK inhibitor is selected from the group consisting of binimetinib, cobimetinib, selumetinib, trametinib, and an analog thereof.

In an aspect of methods provided herein, the tumor cells comprise HSR. In some cases, the method further comprises the step of assessing the tumor cells for a presence, an amount, or a change in the presence or the amount of ecDNA. In some cases, the presence, the amount, or the change in the presence or the amount of ecDNA is assessed using one or more of FISH, whole genome sequencing, whole exome sequencing, targeted panel sequencing, assaying for one or more biomarkers of ecDNA, and assaying for a presence or an amount of an oncogene amplification.

In an aspect of methods provided herein, the RSPA is selected from the group consisting of a RNR inhibitor, an ATR inhibitor, a CHK1 inhibitor, a WEE1 inhibitor, and a PARG inhibitor. In some cases, the RNR inhibitor is selected from the group consisting of 5-chloro-2-(n-((1S,2R)-2-(6-fluoro-2,3-dimethylphenyl)-1-(5-oxo-4,5-dihydro-1,3,4-oxadiazol-2-yl)propyl)sulfamoylbenzamide, cladribine, clofarabine, COH29 (N-[4-(3,4-dihydroxyphenyl)-5-phenyl-1,3-thiazol-2-yl]-3,4-dihydroxybenzamide), fludarabine, gemcitabine, hydroxyurea, motexafin gadolinium, tezacitabine, and triapine. In some embodiments, the AIR inhibitor is selected from the group consisting of ART-0380, ATRN-119, ATRN-212, AZ-20, AZZ-6738, BAY-1895344, berzosertib (M-6620, VX-970; VE-822), BKT-300, IMP-9064, M-1774, M-4344 (VX-803), M-6620, nLs-BG-129, NU-6027, RP-3500, and SC-0245. In some cases, the CHK1 inhibitor is selected from the group consisting of AZD-7762, BEBT-260, GDC-0575, LY-2880070, PF-477736, prexasertib (ACR-368), rabusertib (LY-2603618), RG-7602, SCH-900776, SRA737, and XCCS-605B. In some cases, the WEE1 inhibitor is selected from the group consisting of AZD1775 (MK1775), Bos-I, bosutinib, DC-859/A, Debio 0123, IMP7068, NUV-569, PD0166285, PD0407824, SC-0191, SDR-7778, SDR-7995, and ZN-c3. In some cases, the PARG inhibitor is selected from the group consisting of PD00017273, 3-((1-Methyl-1H-pyrazol-4-yl)methyl)-N-(1-methylcyclopropyl)-1-(oxetan-3-ylmethyl)-2,4-dioxo-1,2,3,4-tetrahydroquinazoline-6-sulfonamide, and (R)—N-(1-Cyanocyclopropyl)-1-(5-(difluoromethyl)-1,3,4-thiadiazol-2-yl)-4-(3-methyl-4-(1-methylcyclopropane-1-carbonyl)piperazin-1-yl)-1H-indazole-6-sulfonamide. In some cases, the treatment with the RSPA results in reduced growth of the tumor cells, the size of the tumor cells, or the number of the tumor cells in the subject.

Neuroblastoma

In an aspect, provided herein are methods of treating cancer in a subject, for example methods of treating a neuroblastoma in a subject. In some cases, methods herein comprise treating the subject with a replication stress pathway agent (RSPA). In some cases, the replication stress pathway agent (RSPA) is used in an amount sufficient to induce replication stress in the tumor cells. In some cases, the method further comprises identifying a subject suffering from or diagnosed with a neuroblastoma. In some cases, tumor cells of the neuroblastoma in the subject are resistant, are reduced in responsiveness, or non-responsive to a therapeutic treatment. In some cases, the treatment with a replication stress pathway agent (RSPA) reduces the growth of the tumor cells, the size of the tumor cells, or the number of the tumor cells.

In an aspect of methods provided herein, the tumor cells comprise an amplification of CDK4, MDM2, MYCN, or any combination thereof. In some cases, the tumor cells are upregulated for one or more of a drug efflux pump or a cancer stem cell marker. In some embodiments, the one or more of the drug efflux pump or the cancer stem cell marker comprise ABCB1, ABCC1-ABCC6, ABCC10-ABCC12, ABCG2, BCL-2L1, CD24, CD44, MCL-1, GL1, NOTCH-1, NOTCH-2, NOTCH-3, and SOX2.

In an aspect of methods provided herein, the tumor cells comprise ecDNA. In some cases, the ecDNA comprises nucleic acid encoding CDK4, MDM2, MYCN, a portion thereof, or an amplification thereof. In some cases, the subject has been previously treated or concurrently treated with a standard of care (SOC) therapeutic agent. In some cases, the SOC is selected from the group consisting of cisplatin, carboplatin, cyclophosphamide, doxorubicin, etoposide, vincristine, and an analog thereof. In some cases, the subject has been previously treated or concurrently treated with a therapeutic agent targeted against CDK4, MDM2, or MYCN.

In an aspect of methods provided herein, the tumor cells comprise HSR. In some cases, the method further comprises the step of assessing the tumor cells for a presence, an amount, or change in the presence or the amount of ecDNA. In some embodiments, the presence, the amount, or the change in the presence or the amount of ecDNA is assessed using one or more of FISH, whole genome sequencing, whole exome sequencing, targeted panel sequencing, assaying for one or more biomarkers of ecDNA, and assaying for a presence or an amount of an oncogene amplification. In some cases, the RSPA is selected from the group consisting of a RNR inhibitor, an ATR inhibitor, a CHK1 inhibitor, a WEE1 inhibitor, and a PARG inhibitor. In some cases, the RNR inhibitor is selected from the group consisting of 5-chloro-2-(n-((1S,2R)-2-(6-fluoro-2,3-dimethylphenyl)-1-(5-oxo-4,5-dihydro-1,3,4-oxadiazol-2-yl)propyl)sulfamoyl)benzamide, cladribine, clofarabine, COH29 (N-[4-(3,4-dihydroxyphenyl)-5-phenyl-1,3-thiazol-2-yl]-3,4-dihydroxybenzamide), fludarabine, gemcitabine, hydroxyurea, motexafin gadolinium, tezacitabine, and triapine. In some cases, the ATR inhibitor is selected from the group consisting of ART-0380, ATRN-119, ATRN-212, AZ-20, AZZ-6738, BAY-1895344, berzosertib (M-6620, VX-970; VE-822), BKT-300, IMP-9064, M-1774, M-4344 (VX-803), M-6620, nLs-BG-129, NU-6027, RP-3500, and SC-0245. In some cases, the CHK1 inhibitor is selected from the group consisting of AZD-7762, BEBT-260, GDC-0575, LY-2880070, PF-477736, prexasertib (ACR-368), rabusertib (LY-2603618), RG-7602, SCH-900776, SRA737, and XCCS-605B. In some cases, the WEE1 inhibitor is selected from the group consisting of AZD1775 (MK1775), Bos-I, bosutinib, DC-859/A, Debio 0123, IMP7068, NUV-569, PD0166285, PD0407824, SC-0191, SDR-7778, SDR-7995, and ZN-c3. In some cases, the PARG inhibitor is selected from the group consisting of PD00017273, 3-((1-Methyl-1H-pyrazol-4-yl)methyl)-N-(1-methylcyclopropyl)-1-(oxetan-3-ylmethyl)-2,4-dioxo-1,2,3,4-tetrahydroquinazoline-6-sulfonamide, and (R)—N-(1-Cyanocyclopropyl)-1-(5-(difluoromethyl)-1,3,4-thiadiazol-2-yl)-4-(3-methyl-4-(1-methylcyclopropane-1-carbonyl)piperazin-1-yl)-1H-indazole-6-sulfonamide. In some cases, the treatment with the RSPA results in reduced growth of the tumor cells, the size of the tumor cells, or the number of the tumor cells in the subject.

Gastric Cancer

In an aspect, provided herein are methods of treating cancer in a subject, for example methods of treating a gastric cancer in a subject. In some cases, methods herein comprise treating the subject with a replication stress pathway agent (RSPA). In some cases, the replication stress pathway agent (RSPA) is used in an amount sufficient to induce replication stress in the tumor cells. In some cases, the method further comprises identifying a subject suffering from or diagnosed with a gastric cancer. In some cases, tumor cells of the gastric cancer in the subject are resistant, are reduced in responsiveness, or non-responsive to a therapeutic treatment. In some cases, the treatment with a replication stress pathway agent (RSPA) reduces the growth of the tumor cells, the size of the tumor cells, or the number of the tumor cells.

In an aspect of methods provided herein, the tumor cells comprise an amplification of FGFR2. In some cases, the tumor cells comprise ecDNA. In some cases, the ecDNA comprises nucleic acid encoding FGFR2, a portion thereof, or an amplification thereof. In some cases, the subject has been previously treated or concurrently treated with an FGFR2 inhibitor. In some cases, the FGFR2 inhibitor is selected from the group consisting of erdafitinib, infigratinib, KIN-3248, erdafitinib, pemigatinib, RLY-4008, TYRA-200, and an analog thereof.

In an aspect of methods provided herein, the tumor cells comprise HSR. In some cases, the method further comprises the step of assessing the tumor cells for a presence, an amount, or change in the presence or the amount of ecDNA. In some cases, the presence, the amount, or the change in the presence or the amount of ecDNA is assessed using one or more of FISH, whole genome sequencing, whole exome sequencing, targeted panel sequencing, assaying for one or more biomarkers of ecDNA, and assaying for the presence or amount of an oncogene amplification.

In an aspect of methods provided herein, the RSPA is selected from the group consisting of a RNR inhibitor, an ATR inhibitor, a CHK1 inhibitor, a WEE1 inhibitor, and a PARG inhibitor. In some cases, the RNR inhibitor is selected from the group consisting of 5-chloro-2-(n-((1S,2R)-2-(6-fluoro-2,3-dimethylphenyl)-1-(5-oxo-4,5-dihydro-1,3,4-oxadiazol-2-yl)propyl)sulfamoyl)benzamide, cladribine, clofarabine, COH29 (N-[4-(3,4-dihydroxyphenyl)-5-phenyl-1,3-thiazol-2-yl]-3,4-dihydroxybenzamide), fludarabine, gemcitabine, hydroxyurea, motexafin gadolinium, tezacitabine, and triapine. In some cases, the ATR inhibitor is selected from the group consisting of ART-0380, ATRN-119, ATRN-212, AZ-20, AZZ-6738, BAY-1895344, berzosertib (M-6620, VX-970; VE-822), BKT-300, IMP-9064, M-1774, M-4344 (VX-803), M-6620, nLs-BG-129, NU-6027, RP-3500, and SC-0245. In some cases, the CHK1 inhibitor is selected from the group consisting of AZD-7762, BEBT-260, GDC-0575, LY-2880070, PF-477736, prexasertib (ACR-368), rabusertib (LY-2603618), RG-7602, SCH-900776, SRA737, and XCCS-605B. In some cases, the WEE1 inhibitor is selected from the group consisting of AZD1775 (MK1775), Bos-I, bosutinib, DC-859/A, Debio 0123, IMP7068, NUV-569, PD0166285, PD0407824, SC-0191, SDR-7778, SDR-7995, and ZN-c3. In some cases, the PARG inhibitor is selected from the group consisting of PD00017273, 3-((1-Methyl-1H-pyrazol-4-yl)methyl)-N-(1-methylcyclopropyl)-1-(oxetan-3-ylmethyl)-2,4-dioxo-1,2,3,4-tetrahydroquinazoline-6-sulfonamide, and (R)—N-(1-Cyanocyclopropyl)-1-(5-(difluoromethyl)-1,3,4-thiadiazol-2-yl)-4-(3-methyl-4-(1-methylcyclopropane-1-carbonyl)piperazin-1-yl)-1H-indazole-6-sulfonamide. In some cases, the treatment with the RSPA results in reduced growth of the tumor cells, the size of the tumor cells, or the number of the tumor cells in the subject.

Esophageal Cancer

In an aspect, provided herein are methods of treating cancer in a subject, for example methods of treating an esophageal cancer in a subject. In some cases, methods herein comprise treating the subject with a replication stress pathway agent (RSPA). In some cases, the replication stress pathway agent (RSPA) is used in an amount sufficient to induce replication stress in the tumor cells. In some cases, the method further comprises identifying a subject suffering from or diagnosed with an esophageal cancer. In some cases, tumor cells of the esophageal cancer in the subject are resistant, are reduced in responsiveness, or non-responsive to a therapeutic treatment. In some cases, the treatment with a replication stress pathway agent (RSPA) reduces the growth of the tumor cells, the size of the tumor cells, or the number of the tumor cells.

In an aspect of methods provided herein, the tumor cells comprise an amplification of EGFR or MDM2. In some cases, the tumor cells comprise ecDNA. In some cases, the ecDNA comprises nucleic acid encoding EGFR, MDM2, a portion thereof, or an amplification thereof. In some cases, the subject has been previously treated or concurrently treated with an EGFR inhibitor or an MDM2 inhibitor. In some cases, EGFR inhibitor is selected from the group consisting of erlotinib, gefitinib, osimertinib, and an analog thereof. In some cases, EGFR inhibitor is a biologic that binds to and/or inhibits EGFR selected from the group consisting of an antibody, antibody drug conjugate and antigen binding fragment. In some embodiments, the antibody for EGFR is selected from cetuximab, panitumumab, nimotuzumab, and necitumumab. In some cases, the MDM2 inhibitor is selected from the group consisting of AD-021.32, ALRN-6924, AM-8533, AMG232, ASTX-295, B1907828, HDM201, KT-253, RG-738, MI-43, Milademetan (RAIN32), serdemetan, SIL-43, and PXN527.

In an aspect of methods provided herein, the tumor cells comprise HSR. In some cases, the method further comprises the step of assessing the tumor cells for a presence, an amount, or change in the presence or the amount of ecDNA. In some cases, the presence, the amount, or the change in the presence or the amount of ecDNA is assessed using one or more of FISH, whole genome sequencing, whole exome sequencing, targeted panel sequencing, assaying for one or more biomarkers of ecDNA, and assaying for a presence or an amount of an oncogene amplification.

In an aspect of methods provided herein, the RSPA is selected from the group consisting of a RNR inhibitor, an ATR inhibitor, a CHK1 inhibitor, a WEE1 inhibitor, and a PARG inhibitor. In some cases, the RNR inhibitor is selected from the group consisting of 5-chloro-2-(n-((1S,2R)-2-(6-fluoro-2,3-dimethylphenyl)-1-(5-oxo-4,5-dihydro-1,3,4-oxadiazol-2-yl)propyl)sulfamoyl)benzamide, cladribine, clofarabine, COH29 (N-[4-(3,4-dihydroxyphenyl)-5-phenyl-1,3-thiazol-2-yl]-3,4-dihydroxybenzamide), fludarabine, gemcitabine, hydroxyurea, motexafin gadolinium, tezacitabine, and triapine. In some cases, the ATR inhibitor is selected from the group consisting of ART-0380, ATRN-119, ATRN-212, AZ-20, AZZ-6738, BAY-1895344, berzosertib (M-6620, VX-970; VE-822), BKT-300, IMP-9064, M-1774, M-4344 (VX-803), M-6620, nLs-BG-129, NU-6027, RP-3500, and SC-0245. In some cases, the CHK1 inhibitor is selected from the group consisting of AZD-7762, BEBT-260, GDC-0575, LY-2880070, PF-477736, prexasertib (ACR-368), rabusertib (LY-2603618), RG-7602, SCH-900776, SRA737, and XCCS-605B. In some cases, the WEE1 inhibitor is selected from the group consisting of AZD1775 (MK1775), Bos-I, bosutinib, DC-859/A, Debio 0123, IMP7068, NUV-569, PD0166285, PD0407824, SC-0191, SDR-7778, SDR-7995, and ZN-c3. In some cases, the PARG inhibitor is selected from the group consisting of PD00017273, 3-((1-Methyl-1H-pyrazol-4-yl)methyl)-N-(1-methylcyclopropyl)-1-(oxetan-3-ylmethyl)-2,4-dioxo-1,2,3,4-tetrahydroquinazoline-6-sulfonamide, and (R)—N-(1-Cyanocyclopropyl)-1-(5-(difluoromethyl)-1,3,4-thiadiazol-2-yl)-4-(3-methyl-4-(1-methylcyclopropane-1-carbonyl)piperazin-1-yl)-1H-indazole-6-sulfonamide. In some cases, the treatment with the RSPA results in reduced growth of the tumor cells, the size of the tumor cells, or the number of the tumor cells in the subject.

Glioblastoma

In an aspect, provided herein are methods of treating cancer in a subject, for example methods of treating a glioblastoma in a subject. In some cases, methods herein comprise treating the subject with a replication stress pathway agent (RSPA). In some cases, the replication stress pathway agent (RSPA) is used in an amount sufficient to induce replication stress in the tumor cells. In some cases, the method further comprises identifying a subject suffering from or diagnosed with a glioblastoma. In some cases, tumor cells of the glioblastoma in the subject are resistant, are reduced in responsiveness, or non-responsive to a therapeutic treatment. In some cases, the treatment with a replication stress pathway agent (RSPA) reduces the growth of the tumor cells, the size of the tumor cells, or the number of the tumor cells.

In an aspect of methods provided herein, the tumor cells comprise an amplification of CDK4, CDK6, and EGFR. In some cases, the tumor cells comprise ecDNA. In some cases, the ecDNA comprises nucleic acid encoding EGFR, a portion thereof, or an amplification thereof. In some cases, the subject has been previously treated or concurrently treated with an EGFR inhibitor. In some cases, the EGFR inhibitor is selected from the group consisting of erlotinib, gefitinib, osimertinib, and an analog thereof. In some cases, EGFR inhibitor is a biologic that binds to and/or inhibits EGFR selected from the group consisting of an antibody, antibody drug conjugate and antigen binding fragment. In some embodiments, antibody for EGFR is selected from cetuximab, panitumumab, nimotuzumab, and necitumumab.

In an aspect of methods provided herein, the tumor cells comprise HSR. In some cases, the method further comprises the step of assessing the tumor cells for a presence, an amount, or change in the presence or the amount of ecDNA. In some cases, the presence, the amount, or the change in the presence or the amount of ecDNA is assessed using one or more of FISH, whole genome sequencing, whole exome sequencing, targeted panel sequencing, assaying for one or more biomarkers of ecDNA, and assaying for a presence or an amount of an oncogene amplification.

In an aspect of methods provided herein, the RSPA is selected from the group consisting of a RNR inhibitor, an ATR inhibitor, a CHK1 inhibitor, a WEE1 inhibitor, and a PARG inhibitor. In some cases, the RNR inhibitor is selected from the group consisting of 5-chloro-2-(n-((1S,2R)-2-(6-fluoro-2,3-dimethylphenyl)-1-(5-oxo-4,5-dihydro-1,3,4-oxadiazol-2-yl)propyl)sulfamoyl)benzamide, cladribine, clofarabine, COH29 (N-[4-(3,4-dihydroxyphenyl)-5-phenyl-1,3-thiazol-2-yl]-3,4-dihydroxybenzamide), fludarabine, gemcitabine, hydroxyurea, motexafin gadolinium, tezacitabine, and triapine. In some cases, the ATR inhibitor is selected from the group consisting of ART-0380, ATRN-119, ATRN-212, AZ-20, AZZ-6738, BAY-1895344, berzosertib (M-6620, VX-970; VE-822), BKT-300, IMP-9064, M-1774, M-4344 (VX-803), M-6620, nLs-BG-129, NU-6027, RP-3500, and SC-0245. In some cases, the CHK1 inhibitor is selected from the group consisting of AZD-7762, BEBT-260, GDC-0575, LY-2880070, PF-477736, prexasertib (ACR-368), rabusertib (LY-2603618), RG-7602, SCH-900776, SRA737, and XCCS-605B. In some cases, the WEE1 inhibitor is selected from the group consisting of AZD1775 (MK1775), Bos-T, bosutinib, DC-859/A, Debio 0123, IMP7068, NUV-569, PD0166285, PD0407824, SC-0191, SDR-7778, SDR-7995, and ZN-c3. In some cases, the PARG inhibitor is selected from the group consisting of PD00017273, 3-((1-Methyl-1H-pyrazol-4-yl)methyl)-N-(1-methylcyclopropyl)-1-(oxetan-3-ylmethyl)-2,4-dioxo-1,2,3,4-tetrahydroquinazoline-6-sulfonamide, and (R)—N-(1-Cyanocyclopropyl)-1-(5-(difluoromethyl)-1,3,4-thiadiazol-2-yl)-4-(3-methyl-4-(1-methylcyclopropane-1-carbonyl)piperazin-1-yl)-1H-indazole-6-sulfonamide. In some cases, the treatment with the RSPA results in reduced growth of the tumor cells, the size of the tumor cells, or the number of the tumor cells in the subject.

Lung Cancer

In an aspect, provided herein are methods of treating cancer in a subject, for example methods of treating a lung cancer in a subject. In some cases, methods herein comprise treating the subject with a replication stress pathway agent (RSPA). In some cases, the replication stress pathway agent (RSPA) is used in an amount sufficient to induce replication stress in the tumor cells. In some cases, the method further comprises identifying a subject suffering from or diagnosed with a lung cancer. In some cases, tumor cells of the glioblastoma in the subject are resistant, are reduced in responsiveness, or non-responsive to a therapeutic treatment. In some cases, the treatment with a replication stress pathway agent (RSPA) reduces the growth of the tumor cells, the size of the tumor cells, or the number of the tumor cells.

In an aspect of methods provided herein, the tumor cells comprise an amplification of EGFR, KRAS, or MYC. In some cases, the tumor cells comprise ecDNA. In some cases, the ecDNA comprises nucleic acid encoding EGFR, KRAS, MYC, a portion thereof, or an amplification thereof.

In an aspect of methods provided herein, the subject has been previously treated or concurrently treated with an EGFR inhibitor, a KRAS inhibitor, or a MYC inhibitor. In some cases, the EGFR inhibitor is selected from the group consisting of erlotinib, gefitinib, osimertinib, and an analog thereof. In some cases, EGFR inhibitor is a biologic that binds to and/or inhibits EGFR selected from the group consisting of an antibody, antibody drug conjugate and antigen binding figment. In some embodiments, antibody for EGFR is selected from cetuximab, panitumumab, nimotuzumab, and necitumumab. In some cases, the KRAS inhibitor is selected from the group consisting of adagrasib, sotorasib, and an analog thereof. In some cases, the KRAS inhibitor is selected from the group consisting of BI 1823911, D-1553, ERAS-3490, GDC-6036, JDQ443, JNJ-74699157 (ARS-3248), LY3499446, LY3537982, and RM-018. In some cases, the subject has been previously treated or concurrently treated with chemotherapy, optionally one or more of paclitaxel, doxorubicin, and vincristine. In some cases, the tumor cells comprise an amino acid mutation in KRAS. In some cases, the tumor cells comprise a G12C mutation in KRAS.

In an aspect of methods provided herein, the tumor cells comprise HSR. In some cases, the method further comprises the step of assessing the tumor cells for a presence, an amount, or change in the presence or the amount of ecDNA. In some cases the presence, the amount, or the change in the presence or the amount of ecDNA is assessed using one or more of FISH, whole genome sequencing, whole exome sequencing, targeted panel sequencing, assaying for one or more biomarkers of ecDNA, and assaying for a presence or an amount of an oncogene amplification.

In an aspect of methods provided herein, the RSPA is selected from the group consisting of a RNR inhibitor, an ATR inhibitor, a CHK1 inhibitor, a WEE1 inhibitor, and a PARG inhibitor. In some cases, the RNR inhibitor is selected from the group consisting of 5-chloro-2-(n-((1S,2R)-2(6-fluoro-2,3-dimethylphenyl)-1-(5-oxo-4,5-dihydro-1,3,4-oxadiazol-2-yl)propyl)sulfamoyl)benzamide, cladribine, clofarabine, COH29 (N-[4-(3,4-dihydroxyphenyl)-5-phenyl-1,3-thiazol-2-yl]-3,4-diydroxybenzamide), fludarabine, gemcitabine, hydroxyurea, motexafin gadolinium, tezacitabine, and triapine. In some cases, the ATR inhibitor is selected from the group consisting of ART-0380, ATRN-119, ATRN-212, AZ-20, AZZ-6738, BAY-1895344, berzosertib (M-6620, VX-970; VE-822), BKT-300, IMP-9064, M-1774, M-4344 (VX-803), M-6620, nLs-BG-129, NU-6027, RP-3500, and SC-0245. In some cases, the CHK1 inhibitor is selected from the group consisting of AZD-7762, BEBT-260, GDC-0575, LY-2880070, PF-477736, prexasertib (ACR-368), rabusertib (LY-2603618), RG-7602, SCH-900776, SRA737, and XCCS-605B. In some cases, the WEE1 inhibitor is selected from the group consisting of AZD1775 (MK1775), Bos-I, bosutinib, DC-859/A, Debio 0123, IMP7068, NUV-569, PD0166285, PD0407824, SC-0191, SDR-7778, SDR-7995, and ZN-c3. In some cases, the PARG inhibitor is selected from the group consisting of PD00017273, 3-((1-Methyl-1H-pyrazol-4-yl)methyl)-N-(1-methylcyclopropyl)-1-(oxetan-3-ylmethyl)-2,4-dioxo-1,2,3,4-tetrahydroquinazoline-6-sulfonamide, and (R)—N-(1-Cyanocyclopropyl)-1-(5-(difluoromethyl)-1,3,4-thiadiazol-2-yl)-4-(3-methyl-4-(1-methylcyclopropane-1-carbonyl)piperazin-1-yl)-1H-indazole-6-sulfonamide. In some cases, the treatment with the RSPA results in reduced growth of the tumor cells, the size of the tumor cells, or the number of the tumor cells in the subject.

In another aspect, provided herein are methods for identifying a subject responsive to a replication stress pathway agent (RSPA). In some cases, the method comprises preparing a first sample and a second sample from a tumor or tumor cell acquired from the subject. In some cases, the first sample and the second sample are derived from different time points. In some cases, a presence and/or a level of ecDNA in the first sample and the second sample is assessed. In some cases, the presence and/or the level of ecDNA of the second sample is compared to the first sample. In some cases, the subject is treated with the RSPA based on a differential the presence and/or the level of ecDNA of the second sample as compared to the first sample.

In an aspect of methods provided herein, the first sample is acquired prior to treatment with a first therapeutic agent. In some cases, the second sample is acquired during the course of treatment or subsequent to treatment with the first therapeutic agent. In some cases, the subject is treated with the RSPA if the level of ecDNA is increased in the second sample as compared to the first sample. In some cases, the second sample comprises an alteration in the structure of the ecDNA as compared to the first sample. In some cases, the alteration comprises an amplification of a gene and/or an oncogene on ecDNA of the second sample as compared to the first sample.

In an aspect of methods provided herein, the tumor or tumor cell is a melanoma. In some cases, the tumor or tumor cells of the second sample are reduced in responsiveness to a mitogen-activated protein kinase (MAPK) inhibitor. In some cases, the tumor or tumor cell is a melanoma and the oncogene is BRAF. In some cases, the BRAF has an amino acid substitution in codon 600 of BRAF. In some cases, the subject has been previously treated or concurrently treated with a BRAF inhibitor, a MEK inhibitor, or a combination thereof. In some cases, the BRAF inhibitor is selected from the group consisting of dabrafenib, emurafenib, encorafenib, KIN-2787, vemurafenib, and an analog thereof. In some cases, the MEK inhibitor is selected from the group consisting of binimetinib, cobimetinib, selumetinib, trametinib, and an analog thereof.

In an aspect of methods provided herein, the tumor or tumor cell is a neuroblastoma. In some cases, the tumor or tumor cells of the second sample are reduced in responsiveness to a standard of care treatment. In some cases, the tumor or tumor cell is a neuroblastoma and the oncogene is CDK4, MDM2, MYCN, or a portion thereof. In some cases, the subject has been previously treated or concurrently treated with a standard of care (SOC) therapeutic agent. In some cases, the standard of care (SOC) therapeutic agent is selected from the group consisting of cisplatin, carboplatin, cyclophosphamide, doxorubicin, etoposide, vincristine, and an analog thereof. In some cases, the subject has been previously treated or concurrently treated with a therapeutic agent targeted against CDK4, MDM2, or MYCN.

In an aspect of methods provided herein, the tumor or tumor cell is a gastric cancer. In some cases, the tumor or tumor cells of the second sample are reduced in responsiveness to a therapeutic agent directed against FGFR2. In some cases, the tumor or tumor cell is a gastric cancer and the oncogene is FGFR2. In some cases, the subject has been previously treated or concurrently treated with an FGFR2 inhibitor. In some cases, the FGFR2 inhibitor is selected from the group consisting of erdafitinib, infigratinib, KIN-3248, pemigatinib, RLY-4008, TYRA-200, and an analog thereof.

In an aspect of methods provided herein, the tumor or tumor cell is an esophageal cancer and wherein the tumor or tumor cells of the second sample are reduced in responsiveness to a therapeutic agent directed against EGFR or MDM2. In some cases, the tumor or tumor cell is an esophageal cancer and the oncogene is EGFR or MDM2. In some cases, the subject has been previously treated or concurrently treated with an EGFR inhibitor or an MDM2 inhibitor. In some cases, the EGFR inhibitor is selected from the group consisting of erlotinib, gefitinib, osimertinib, and an analog thereof. In some cases, EGFR inhibitor is a biologic that binds to and/or inhibits EGFR selected from the group consisting of an antibody, antibody drug conjugate and antigen binding fragment. In some cases, the MDM2 inhibitor is selected from the group consisting of AD-021.32, ALRN-6924, AM-8533, AMG232, ASTX-295, B1907828, HDM201, KT-253, RG-738, MI-43, Milademetan (RAIN32), serdemetan, SIL-43, and PXN527.

In an aspect of methods provided herein, the tumor or tumor cell is a glioblastoma. In some cases, the tumor or tumor cells of the second sample are reduced in responsiveness to a therapeutic agent directed against CDK4, CDK6, or EGFR. In some cases, the tumor or tumor cell is a glioblastoma and the oncogene is EGFR. In some cases, the subject has been previously treated or concurrently treated with an EGFR inhibitor. In some cases, the EFGR inhibitor is selected from the group consisting of erlotinib, gefitinib, osimertinib, and an analog thereof.

In an aspect of methods provided herein, the tumor or tumor cell is a lung cancer and wherein the tumor or tumor cells of the second sample are reduced in responsiveness to a therapeutic agent directed against EGFR, KRAS, or MYC. In some cases, the tumor or tumor cell is a lung cancer and the oncogene is EGFR, KRAS, or MYC. In some cases, the subject has been previously treated or concurrently treated with an EGFR inhibitor, a KRAS inhibitor, or a MYC inhibitor. In some cases, the EFGR inhibitor is selected from the group consisting of erlotinib, gefitinib, osimertinib, and an analog thereof. In some cases, the KRAS inhibitor is selected from the group consisting of adagrasib, sotorasib, and an analog thereof. In some cases, the KRAS inhibitor is selected from the group consisting of BI 1823911, D-1553, ERAS-3490, GDC-6036, JDQ443, JNJ-74699157 (ARS-3248), LY3499446, LY3537982, and RM-018. In some cases, the subject has been previously treated or concurrently treated with chemotherapy, optionally one or more of paclitaxel, doxorubicin, and vincristine. In some cases, the subject harbors a G12C mutation in KRAS. In some cases, the step of assessing comprises one or more of FISH, whole genome sequencing, whole exome sequencing, targeted panel sequencing, assaying for one or more biomarkers of ecDNA, and assaying for a presence or an amount of an oncogene amplification. In some cases, the RSPA is selected from the group consisting of a RNR inhibitor, an ATR inhibitor, a CHK1 inhibitor, a WEE1 inhibitor, and a PARG inhibitor. In some cases, the structure of ecDNA is assessed with Amplicon Architect.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Certain Definitions

As used herein the term “about” or “approximately” means within an acceptable error range for the particular value as determined by one of ordinary skill in the art, which can depend in part on how the value is measured or determined, i.e., the limitations of the measurement system. For example, “about” can mean within 1 or more than 1 standard deviation, per the practice in the art. As another example, “about” can mean a range of up to 20%, up to 10%, up to 5%, or up to 1% of a given value. With respect to biological systems or processes, the term “about” can mean within an order of magnitude, such as within 5-fold or within 2-fold of a value. Where particular values are described in the application and claims, unless otherwise stated, the term “about” means within an acceptable error range for the particular value.

The term “subject,” as used herein, generally refers to a vertebrate, such as a mammal (e.g., a human). Mammals include, but are not limited to, murines, simians, humans, farm animals, sport animals, and pets (e.g., a dog or a cat). Tissues, cells, and their progeny of a biological entity obtained in vivo or cultured in vitro are also encompassed. In some embodiments, the subject is a patient. In some embodiments, the subject is symptomatic with respect to a disease (e.g., cancer). Alternatively, in some cases, the subject is asymptomatic with respect to the disease. In some cases, the subject does not have the disease.

The term “biological sample,” as used herein, generally refers to a sample derived from or obtained from a subject, such as a mammal (e.g., a human). Biological samples are contemplated to include but are not limited to, hair, fingernails, skin, sweat, tears, ocular fluids, nasal swab or nasopharyngeal wash, sputum, throat swab, saliva, mucus, blood, serum, plasma, placental fluid, amniotic fluid, cord blood, emphatic fluids, cavity fluids, earwax, oil, glandular secretions, bile, lymph, pus, microbiota, meconium, breast milk, bone marrow, bone, CNS tissue, cerebrospinal fluid, adipose tissue, synovial fluid, stool, gastric fluid, urine, semen, vaginal secretions, stomach, small intestine, large intestine, rectum, pancreas, liver, kidney, bladder, lung and other tissues and fluids derived from or obtained from a subject.

The term “treating” as used herein, generally refers to administering an agent, or carrying out a procedure, for the purposes of obtaining an effect. In some cases, the effect is prophylactic in terms of completely or partially preventing a disease or symptom thereof and/or is therapeutic in terms of effecting a partial or complete cure for a disease and/or one or more symptoms of the disease. “Treatment,” as used herein, can include treatment of a tumor in a mammal, particularly in a human, and includes: (a) preventing the disease or a symptom of a disease from occurring in a subject which can be predisposed to the disease but has not yet been diagnosed as having it (e.g., including diseases that can be associated with or caused by a primary disease; (b) inhibiting the disease, i.e., arresting its development; and (c) relieving the disease, i.e., causing regression of the disease. In some cases, treating refers to any indicia of success in the treatment or amelioration or prevention of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters, including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

The term “tumor” or “tumor cells” as used herein, generally refers to cells that grow and divide more than they should or do not die when they should. In some cases, tumor cells are present in a solid mass, such as a solid tumor, or in some cases, tumor cells are found in a non-solid form, such as in blood cancers. Tumor or tumor cells also can include metastasis or metastasizing cells, where cancer cells break away from the original (primary) tumor and, in some cases, form a new tumor in other organs or tissues of the body.

The term “oncogene” as used herein, generally refers to a gene that has the potential to cause cancer when inappropriately activated. In tumors or tumor cells, these genes are often mutated to remove negative regulatory domains or expressed at high levels.

The term “ecDNA signature” as used herein, generally refers to one or more characteristics common to tumors or tumor cells that are ecDNA+. In some cases, the ecDNA signature is selected from the group consisting of a gene amplification; a p53 loss of function mutation; absence of microsatellite instability (MSI-H); a low level of PD-L1 expression; a low level of tumor inflammation signature (TIS); a low level of tumor mutational burden (TMB); an increased frequency of allele substitutions, insertions, or deletions (indels); and any combination thereof. In some cases, ecDNA signature includes a detection or identification of ecDNA using an imaging technology. In some cases, ecDNA signature does not include any imaging or direct detection of ecDNA.

The terms “replication stress pathway agent,” “RSPA,” “replication stress pathway inhibitor,” and “RS pathway inhibitor” as used herein, generally refer to an agent that causes replication stress in a cell, such as a tumor cell. In some cases, the RSPA is an inhibitor of a replication stress pathway component, where inhibition increases replication stress. Replication stress as used herein refers to a stress that affects DNA replication and/or DNA synthesis and can include but is not limited to the slowing or stalling of replication fork progression and/or interference with DNA synthesis. Exemplary replication stress pathway agents include but are not limited to agents that inhibit RNR (ribonucleotide reductase), CHK1 (checkpoint kinase 1), ATR (Rad3-related protein), WEE1, E2F, PARG (poly(ADP ribose) glycohydrolase), or RRM2 (ribonucleotide reductase regulatory subunit 2).

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### EXAMPLES

The following examples are given for the purpose of illustrating various embodiments of the present disclosure and are not meant to limit the disclosure herein in any fashion. The present examples, along with the methods described herein are presently representative of preferred embodiments, are exemplary, and are not intended as limitations on the scope of the disclosure herein. Changes therein and other uses which are encompassed within the spirit of the disclosure as defined by the scope of the claims will occur to those having ordinary skill in the art.

### Example 1: Treatment of Neuroblastoma in Mice

Focal gene amplification of MYCN is associated with poor outcome and recurrence of aggressive disease in neuroblastoma (NB) patients. In certain instances, NB tumors have harbor amplification of druggable oncogenes such as CDK4 and MDM2. However, these targeted therapies have failed to demonstrate clinical efficacy as monotherapy in the amplified setting.

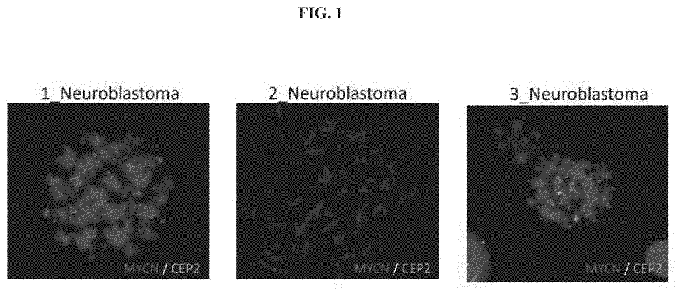

To address this failure, experiments were designed to assess the presence of ecDNA and amplification of oncogenes on such ecDNA and then directly targeting the underlying ecDNA biology in order to manifest deeper and more durable responses. ecDNA was assessed in a neuroblastoma cell lines CHP-126, CHP-212, and MCIXC, using FISH staining for the MYC oncogene. As shown in FIG. 1, MYC amplification on ecDNA was present.

Construction of NB DOX-Resistant Cell Lines

Additional NB cell lines are selected to compare lines with MYCN ecDNA+, integrated ecDNA− (HSR) and mixed ecDNA/HSR treated with agents on ecDNA levels, cells are treated with a NB standard of care agent, such as doxorubicin (DOX) or vincristine (VCR). 3-day proliferation assays (e.g., CTG) IC50 of DOX is determined for each selected line. Cell lines are then subsequently assessed in long-term flask experiments under 2 dosing paradigms with DOX (2× iterative step-up from IC50 and single step IC90). Lines that fail to develop resistance within 12 weeks are discontinued. Cell lines are assessed for the presence and amount of ecDNA using whole genome sequencing (WGS) and/or whole exome sequencing (WES), metaphase DAPI/FISH, and cell count/viability. Amplicon Architect is used to further map the structure of ecDNA in the lines.

Standard of Care Agent Impact on ecDNA

To assess the effect of treatment agents on ecDNA levels, the NB ecDNA+ cell lines are treated with a combination of DOX and an ecDNA-directed therapeutic (ecDTx), as well as controls of untreated cells and cells treated with only ecDTx. The treated cell lines are evaluated in a 7-day anti-proliferation assay to compare the ability of the combination of DOX and ecDTx to impact the response to DOX and the sensitivity of the cell lines to the ecDTx after developing resistance to DOX. The cell lines are further assessed using similar combination treatment for abrogation of DOX resistance formation in long-team culture. The treated cell lines are evaluated for cell viability, and proliferation. An assessment of the formation and maintenance of ecDNA is performed using qPCR, metaphase/interphase FISH, and WGS.

To further understand the role of ecDNA on tumor growth and proliferation, the single agents and combination are evaluated in an ecDNA+NB patient-derived xenograft (PDX) or NB cell line-derived (CDX) mouse models, including models with MYCN amplified, CDK4 amplified, and MDM2 amplified ecDNA+. The mice are treated with the corresponding therapeutic agent to confirm initial sensitivity of the tumor to the agent and subsequent development of resistance. (DOX for MYCN amplified PDX, CDK4/6 inhibitor for CDK4 amplified PDX, and MDM2 inhibitor for MDM2 amplified PDX).

To evaluate the impact of ecDTx, the PDX mouse models are treated with combination of the corresponding therapeutic agent alone, the ecDTx alone or the combination of the corresponding therapeutic agent and the ecDTx. The treated mice are evaluated for tumor growth, changes in ecDNA amplification and ecDNA structure over a time course (e.g., 0-42 days after drug administration). Additional assays can be included such as changes in phosphorylated Rb levels (for CDK4 amplified PDX) and changes in p53 levels or function (for MDM2 amplified PDX).

### Example 2: Treatment of Gastric Cancer in Mice

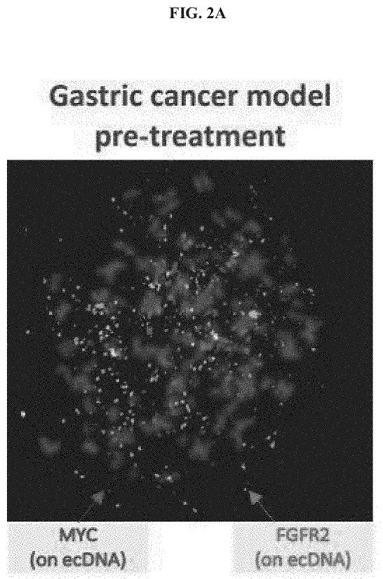

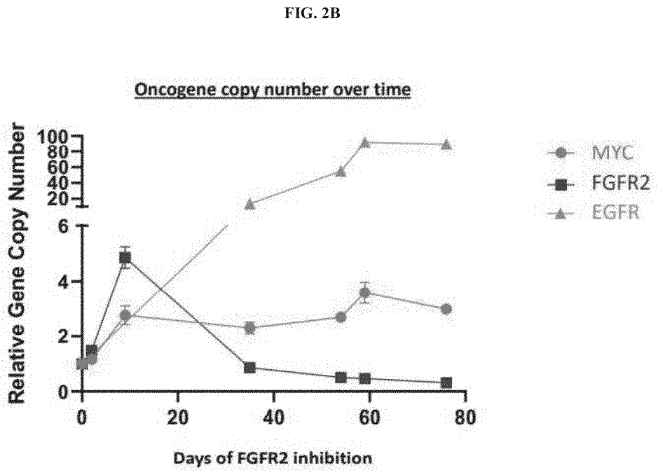

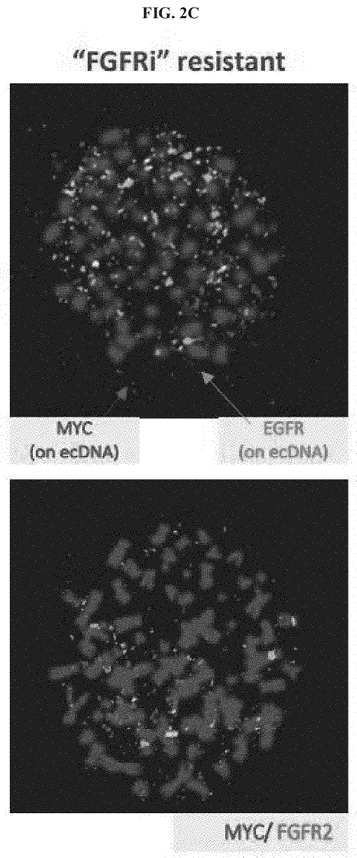

A gastric cancer cell line SNU16 was assessed for the presence of ecDNA using metaphase DAPI/FISH using probes to detect MYC, FGFR2 and EGFR. FIG. 2A shows the DAPI staining along with the identified MYC amplified and FGFR2-amplified ecDNA. SNU16 cells were then treated with the FGFR inhibitor infigratinib over the course of about 80 days. ecDNA was assessed by metaphase DAPI/FISH and relative copy number for MYC, FGFR2 and EGFR was tracked at day 0 and 5 additional time points. Results are shown in FIG. 2B. After about 10 days of treatment with the FGFR inhibitor, the copy number of FGFR2 was reduced dramatically. However, the copy number of MYC and EGFR rapidly increased as shown in the graph in FIG. 2B. FIG. 2C shows by metaphase DAPI/FISH that the EGFR amplification was found on ecDNA.

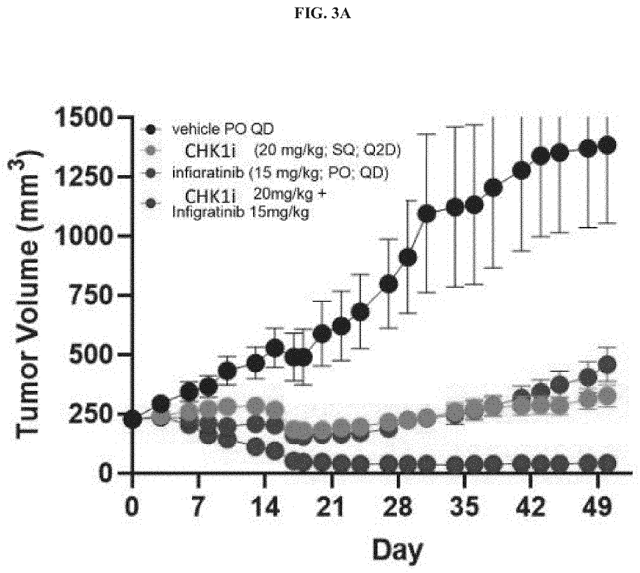

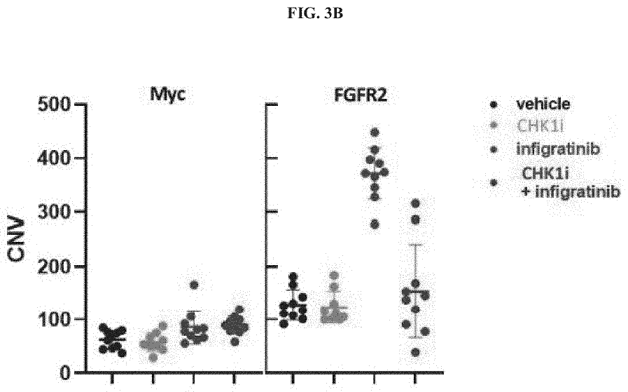

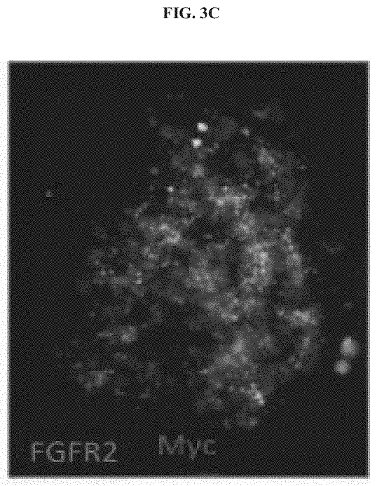

To assess the effect of an ecDTx agent on gastric cancer and ecDNA development, a gastric cancer CDX SNU16 mouse model was treated with vehicle only, with infigratinib alone (15 mg/kg PO;QD), the ecDTx CHK1 inhibitor (20 mg/kg SQ;Q2D) or with a combination of infigratinib (15 mg/kg PO;QD) and the ecDTx CHK1 inhibitor (20 mg/kg SQ;Q2D). Tumor growth in the animals was monitored over 50 days. Results are shown in FIG. 3A. The single agents alone decreased tumor growth as compared to vehicle; however, at the later time points, the trend in the infigratinib alone mice suggested resistance was beginning to develop. In contrast, the combination of the ecDTx with infigratinib decreased tumor growth and maintained its effect over the course of treatment. The effect of the combination was further assessed by evaluating copy number for MYC and FGFR2 in the tumors from the treated mice. As shown in FIG. 3B, the copy number of MYC was relatively similar in the 4 treatment groups. In contrast, the copy number of FGFR2 in the infigratinib alone was dramatically higher than the other groups. FGFR2 amplification was abrogated in the presence of the ecDTx agent alone or when used in combination with infigratinib. The presence of the FGRF2 and MYC amplification on ecDNA was validated by FISH (FIG. 3C).

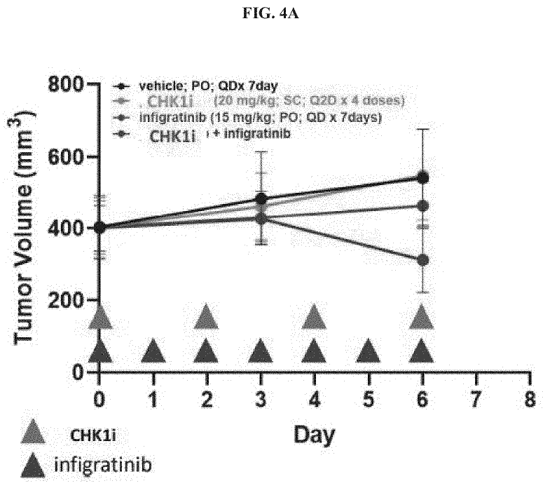

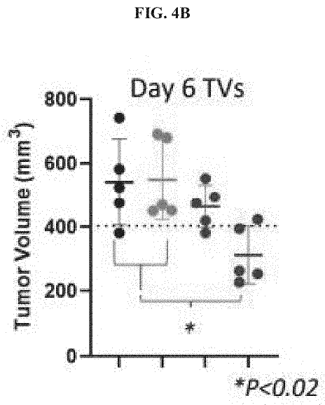

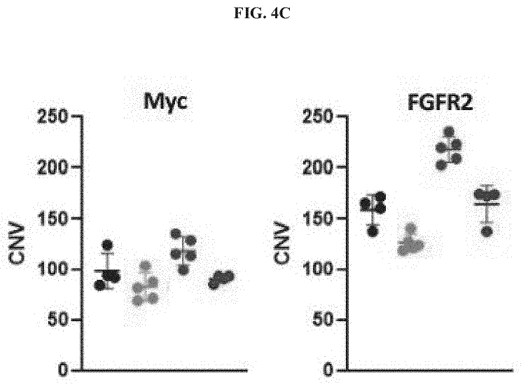

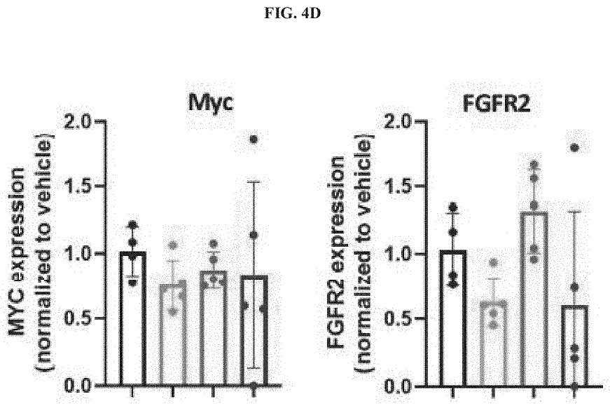

The ecDTx agent was further tested in the gastric cancer CDX model in a 7-day multi-dosing experiment. Mice were treated with vehicle only (PO; QDx 7 days), with infigratinib alone (15 mg/kg PO;QD x 7 days), the ecDTx CHK1 inhibitor (20 mg/kg SC;Q2D x 4 doses) or with a combination of infigratinib and the ecDTx CHK1 inhibitor (same dosing as with individual agents). Tumor volume was measured over the course of the 7 days and shown in FIG. 4A.

Tumor volume at day 6 is shown in the box and whisker graph in FIG. 4B. The combination of ecDTx with infigratinib significantly decreased tumor volume as compared to the vehicle control and the single agents. Copy number for MYC and FGFR2 was evaluated in the tumors from the treated mice. As shown in FIG. 4C, the copy number of MYC was relatively similar in the 4 treatment group, however the copy number of FGFR2 in the infigratinib alone was dramatically higher than the other groups. This increase was abrogated in the presence of the ecDTx agent. MYC and FGFR2 expression was also evaluated in the tumors of the treated mice (FIG. 4D). Similar to the effect on copy number, while MYC expression was similar in the treatment groups, FGFR2 expression was markedly increased in the tumors treated with infigratinib as a single agent. Treatment with the ecDTx in combination abolished the increase in FGFR2 expression seen with infigratinib alone.

### Example 3: Treatment of Sarcoma in Mice

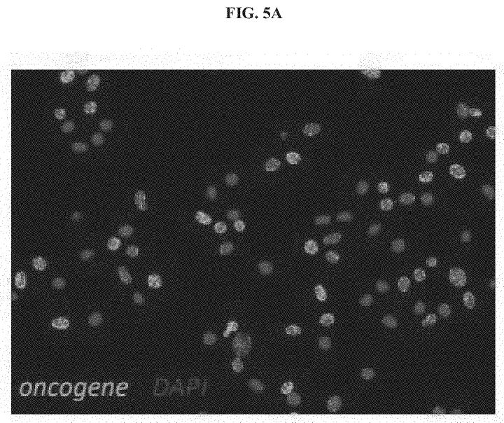

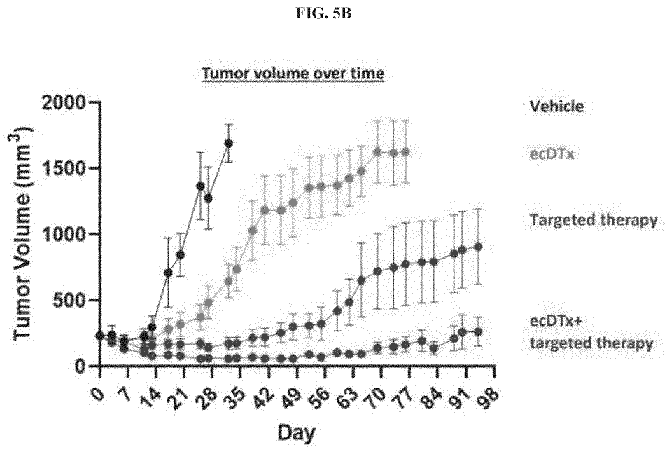

A sarcoma PDX model was evaluated by for the presence of ecDNA using metaphase DAPI/FISH using probes to detect MDM2 and CDK4. Exemplary amplification of MDM2 on ecDNA is shown in FIG. SA. To assess the impact of ecDTx on tumor growth, mice were divided into 4 treatment groups: vehicle only, targeted agent only (Palbociclib, a CDK4/6 inhibitor), ecDTx only, and a combination of targeted agent+ecDTx. Tumor volume was monitored across 90+ days or until tumor volume neared 2000 m3, at which point the mice were euthanized. As shown in FIG. 5B, tumor volume increased rapidly in the vehicle only group. The single agents also showed modest tumor growth inhibition. Surprisingly, the combination of the targeted agent and the ecDTx agent showed a significant and robust anti-tumor activity with tumor regression that was not present with single agent on its own.

### Example 4: Treatment of Glioblastoma in Mice

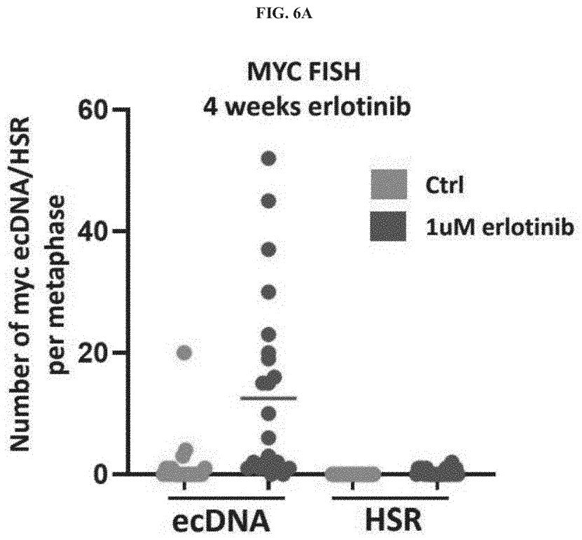

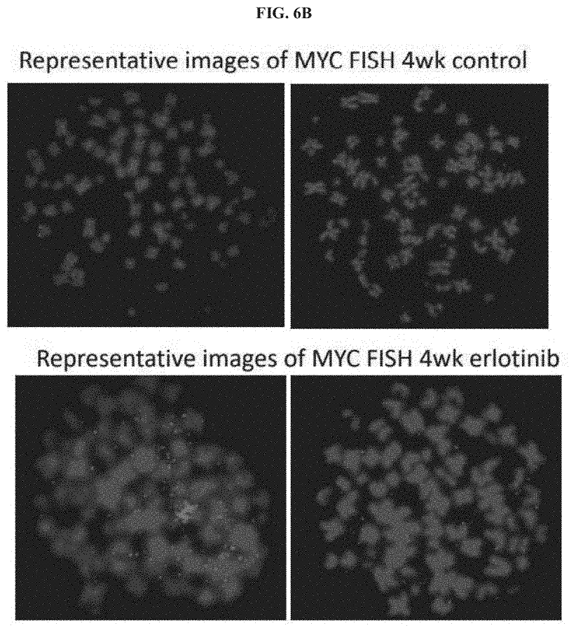

Glioblastoma cell line (GBM39) was assessed for ecDNA in untreated samples and cells treated with the EGFR inhibitor erlotinib (1 uM). Cells were treated for 4 weeks and then assessed by FISH for amplification of MYC ecDNA. Representative FISH images are shown in FIG. 6B. Untreated (control cells) did not show any change in ecDNA staining. Cells treated with the erlotinib developed brightly staining MYC amplification on ecDNA. Amplification of MYC on ecDNA and on HSR was quantified from the FISH images in control and treated cells. As shown in the graph FIG. 6A, the erlotinib-treated cells exhibited a dramatic increase in MYC amplification on ecDNA that was not seen in the control cells. The erlotinib-treated cells did not show any significant difference in MYC amplification on HSR as compared to control cells.

### Example 5: Treatment of Lung Cancer in Mice

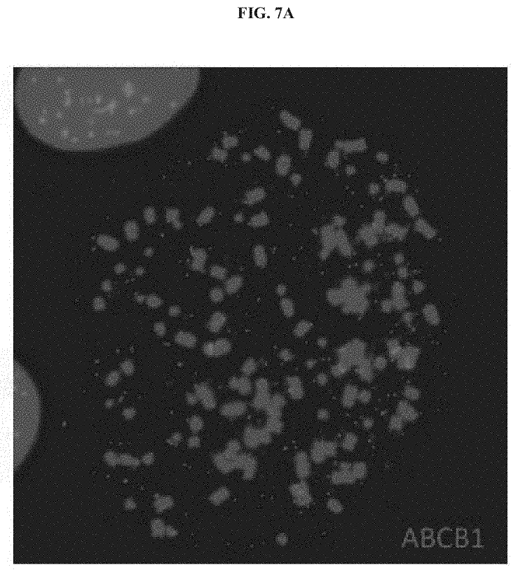

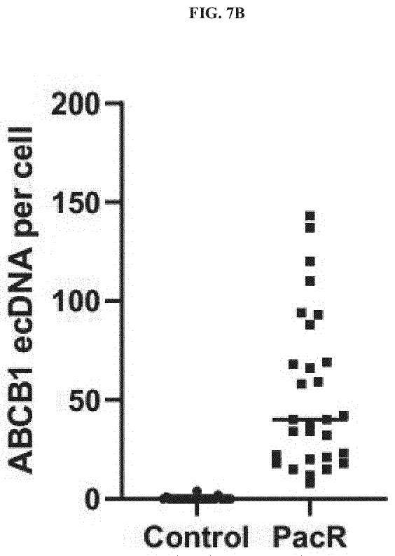

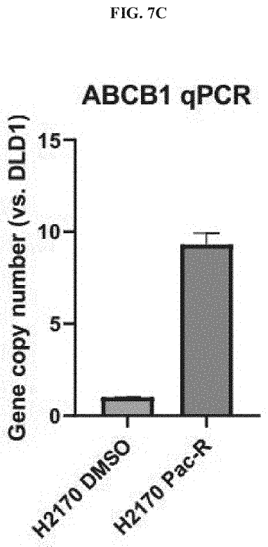

The lung cancer cell line (H2170) was assessed for the presence of oncogene-amplified ecDNA. The cells had high copy numbers of MYC and ERBB2 ecDNA. These amplified ecDNA were present prior to any drug treatment. The cell line was treated with paclitaxel and the presence and level of ecDNA was assessed for ABCB1 transporter amplified ecDNA. A representative FISH image of the treated cells exhibiting ABCB1 transporter amplified ecDNA is shown in FIG. 7A. Copy number of the ABCB1 transporter amplified ecDNA was quantified in untreated controls and paclitaxel-treated cells, shown in FIG. 7B. The drug-treated cells showed a marked increase in ABC1 transporter amplified ecDNA. Gene copy number for the ABCB1 transporter was assessed by qPCR. As shown in FIG. 7C, the paclitaxel-treated cells had a dramatically higher gene copy number as compared to the untreated control.

### Example 6: Treatment of Colorectal Cancer in Mice

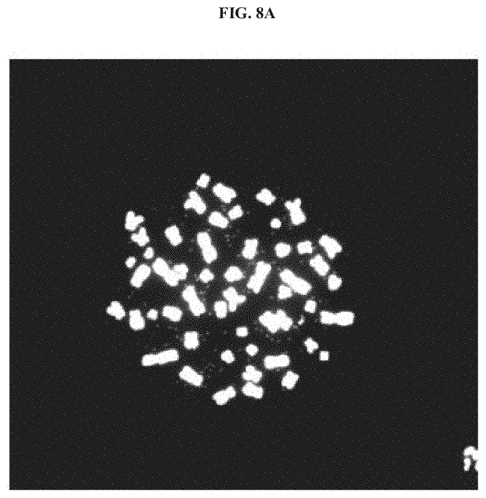

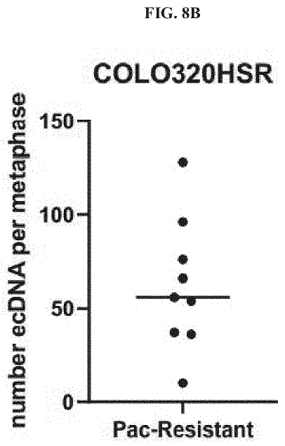

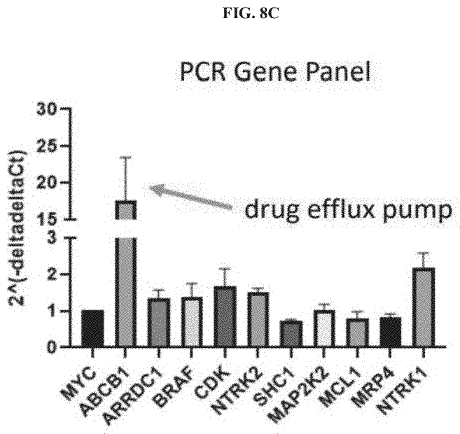

The colorectal cancer line COLO320 HSR was treated with paclitaxel. Cells that grew in the presence of the drug (paclitaxel resistant) were assessed for ecDNA development by metaphase FISH. A representative image of a resistant cell is shown in FIG. 8A. As shown in FIG. 8B, the cells generally exhibited a high copy number of ecDNA harboring the ABCB1 drug efflux pump. PCR with a panel of genes was used to assess the amplification of various genes in the paclitaxel-treated cells. As shown in FIG. 8C, the highest amount of amplification was seen with ABCB1.

### Example 7: Treatment of Melanoma in Mice

Focal gene amplification of BRAFV600E and other genes such as MET have been implicated in BRAF/MEK inhibitor resistance in melanoma patients. Patients develop resistance to BRAFV600E inhibitors vemurafenib and debrafinib alone or when used in combination with the MEK inhibitor selumetinib.

To assess the role and impact of ecDNA in this resistance, experiments were designed to characterize the effects of ecDTx agents in melanoma.

Melanoma cell lines with MAPKi-resistant (e.g., resistant to vemurafenib/selumitinib) ecDNA+ and ecDNA− are treated with ecDTx agents. Cell proliferation and viability are monitored in a 7 day Cell-titerGlo assay. Cell death and apoptosis of the cell lines is assessed using staining assays. ecDNA presence and level is monitored using metaphase FISH and qPCR. A soft-agar colony formation assay is performed in the presence of the vemurafenib and selumitinib, with and without the ecDTx agent. Colony formation (e.g., resistant cells) are compared with and without the ecDTx agent.

The combination of resistance development is assessed as follows: Melanoma cells (starting line ecDNA−) are treated with vemurafenib and selumitinib or with a combination of vemurafenib, selumitinib and ecDTx. Cell proliferation and ecDNA are assayed over long-term culture. To further evaluate the combination treatment, a melanoma PDX model is treated in 4 treatment groups: vehicle alone, ecDTx alone, vemurafenib and selumitinib and ecDTx with vemurafenib and selumitinib. Tumor volume is assayed over a time course of 0-about 50 days. ecDNA is evaluated using FISH and copy number amplification.

### Example 8: Evaluation of ecDNA and ecDTx in Patient-Derived Tumor Models

Patient derived xenograft (PDX) mouse models are created from tumors with and without MAPKi resistance and associated copy number amplification, including wildype melanoma and melanoma harboring the BRAF V600 mutation. WGS is performed on the created PDX models and tumor samples are examined by FISH. Amplicon Architect is run on WGS sequencing data to determine ecDNA status and reconstruct circular architecture of amplicons for ecDNA+ tumors and to validate the ecDNA− status of the control PDX tumors. Mice from each of the PDX models are separated into cohorts for treatment with MAPK inhibitor alone, ecDTx agent alone or the combination of MAPK inhibitor and the ecDTx agent. Tumor growth and ecDNA presence and amount are observed after treatment as described in Example 7.

### Example 9: ecDNA Status and Sensitivity to ecDTx Agent in Cancers

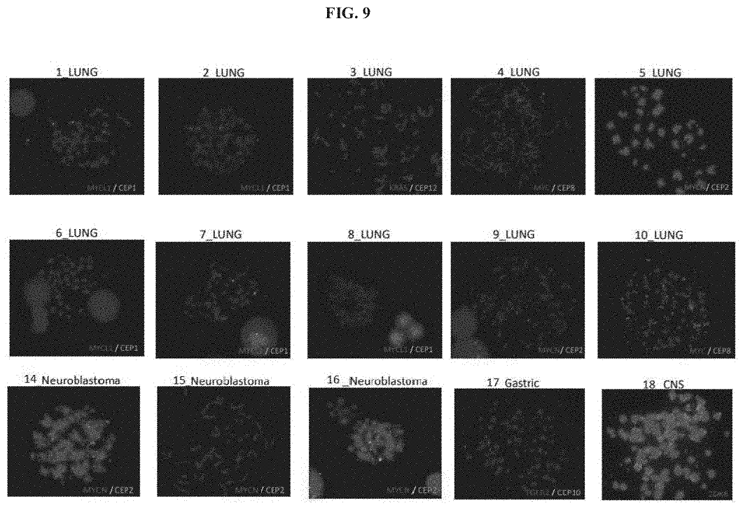

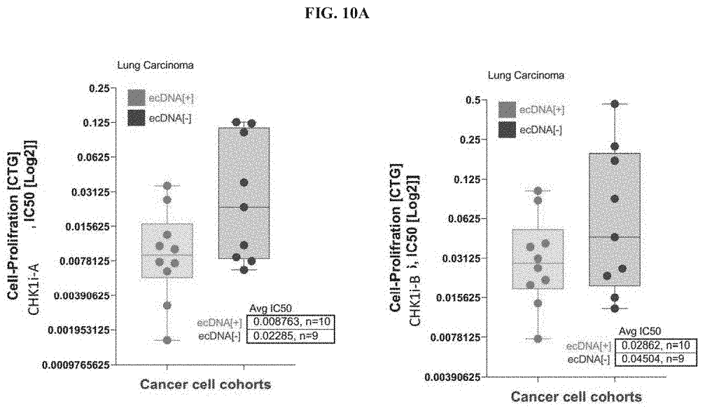

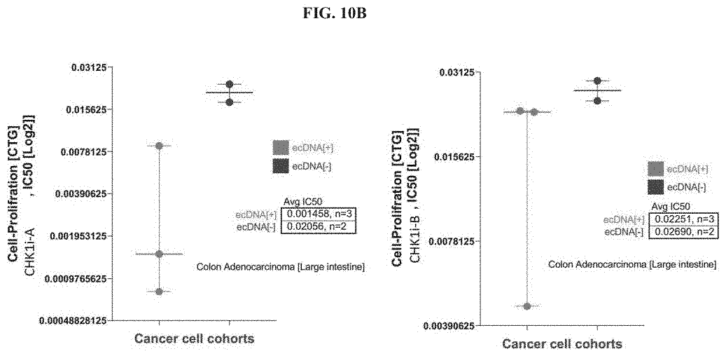

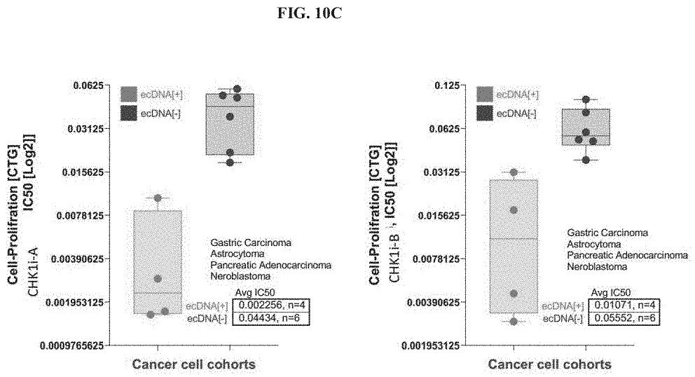

To assess the prevalence of ecDNA in a variety of cancer types, tumor cells were assessed by metaphase FISH using probes for one or more of MYC, MYCL1, MYCN, KRAS, FGFR2, CDK6, CEP1, CEP2, CEP8, CEP10, and CEP12. Results are shown in FIG. 9. ecDNA+ tumor cells from a variety of cancer types were treated with an ecDTx (a CHK1 inhibitor) in a concentration range between 0.0001 and 10 uM. As shown in FIG. 10, ecDTx potently inhibited tumor proliferation of lung carcinoma, colon adenocarcinoma, gastric carcinoma, astrocytoma, pancreatic adenocarcinoma, and neuroblastoma in the ecDNA+ cells.

### Example 10: Gene Amplification and Sensitivity to ecDtx Agent

To further assess the relationship of gene amplification and replication stress, cell lines were profiled for gene amplification. Amplification status was assessed through a combination of copy number calling from next generation sequencing data and cytogenic/FISH analysis of the particular cell line. Oncogene amplifications was defined a >8 DNA copies/gene. A subset of the cell lines was also profiled for replication stress response by measuring protein expression by western blotting for replication stress markers p-RPA and p-CHK1 and by foci formation assessed by immunofluorescence microscopy.

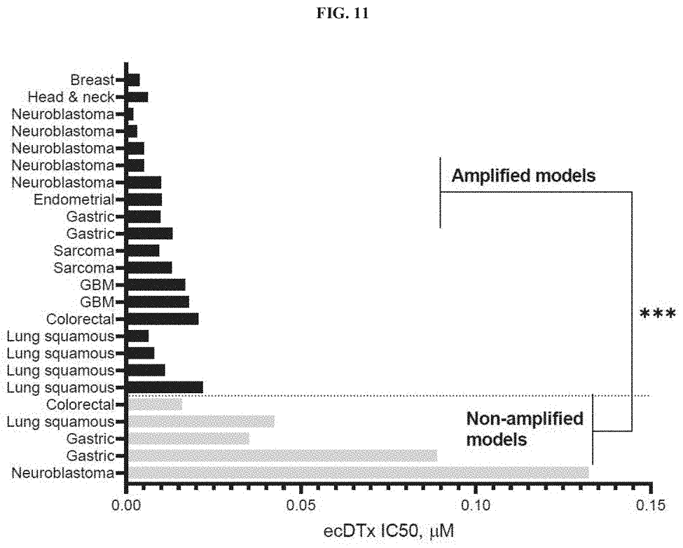

FIG. 11 shows the comparison of the cell lines grouped by cancer type. Cell lines with amplified oncogenes are shown in black bars. Cell lines which lacked oncogene amplification are shown in gray. Each of the cell lines was tested for sensitivity to a CHK1 inhibitor, CHK1 i-C, in a 7-day proliferation assay. The length of the bar along the x-axis indicates the IC50. A dose-dependent enhanced sensitivity was observed at markedly lower drug concentrations of CHK1-C in cell lines with gene amplification, resulting in significantly lowered TC50 values (***p-value>0.001) when compared to cell lines with no identified amplification. Significance between the two groups was determined using Two-tailed Mann Whitney test in GraphPad Prism software. The more sensitive lines also tended to be those displaying increased replication stress response.

While preferred embodiments of the present invention have been shown and described herein, it will be obvious to those skilled in the art that such embodiments are provided by way of example only. Numerous variations, changes, and substitutions will occur to those skilled in the art without departing from the invention. It should be understood that various alternatives to the embodiments described herein may be employed. It is intended that the following claims define the scope of embodiments and that methods and structures within the scope of these claims and their equivalents be covered thereby.

1. A method for treating a cancer comprising:
a) identifying a subject suffering from or diagnosed with a cancer, wherein tumor cells of the cancer are resistant, are reduced in responsiveness, or non-responsive to a therapeutic treatment;
b) treating the subject with a replication stress pathway agent (RSPA) in an amount sufficient to induce replication stress in the tumor cells; and
c) whereby the growth of the tumor cells, the size of the tumor cells, or the number of the tumor cells is reduced,
wherein the cancer is selected from the group consisting of melanoma, neuroblastoma, esophageal cancer, glioblastoma, and lung cancer.

2.-35. (canceled)

36. A method for treating a gastric cancer comprising:
a) identifying a subject suffering from or diagnosed with the gastric cancer, wherein tumor cells of the gastric cancer are resistant, are reduced in responsiveness, or non-responsive to a therapeutic treatment;
b) treating the subject with a replication stress pathway agent (RSPA) in an amount sufficient to induce replication stress in the tumor cells; and
c) whereby the growth of the tumor cells, the size of the tumor cells, or the number of the tumor cells is reduced.

37. The method of claim 36, wherein the tumor cells comprise an amplification of FGFR2.

38. The method of claim 36, wherein the tumor cells comprise ecDNA.

39. The method of claim 38, wherein the ecDNA comprises nucleic acid encoding FGFR2, a portion thereof, or an amplification thereof.

40. The method of claim 36, wherein the subject has been previously treated or concurrently treated with an FGFR2 inhibitor.

41. The method of claim 40, wherein the FGFR2 inhibitor is selected from the group consisting of erdafitinib, infigratinib, KIN-3248, erdafitinib, pemigatinib, RLY-4008, TYRA-200, and an analog thereof.

42. The method of claim 36, wherein the tumor cells comprise HSR.

43. The method of claim 36, wherein the method further comprises the step of assessing the tumor cells for a presence, an amount, or a change in the presence or the amount of ecDNA.

44. The method of claim 43, wherein the step of assessing comprises one or more of FISH, whole genome sequencing, whole exome sequencing, targeted panel sequencing, assaying for one or more biomarkers of ecDNA, and assaying for a presence or an amount of an oncogene amplification.

45. The method of claim 36, wherein the RSPA is selected from the group consisting of a RNR inhibitor, an ATR inhibitor, a CHK1 inhibitor, a WEE1 inhibitor, and a PARG inhibitor.

46. The method of claim 45, wherein the RNR inhibitor is selected from the group consisting of 5-chloro-2-(n-((1S,2R)-2-(6-fluoro-2,3-dimethylphenyl)-1-(5-oxo-4,5-dihydro-1,3,4-oxadiazol-2-yl)propyl)sulfamoyl)benzamide, cladribine, clofarabine, COH29 (N-[4-(3,4-dihydroxyphenyl)-5-phenyl-1,3-thiazol-2-yl]-3,4-dihydroxybenzamide), fludarabine, gemcitabine, hydroxyurea, motexafin gadolinium, tezacitabine, and triapine.

47. The method of claim 45, wherein the ATR inhibitor is selected from the group consisting of ART-0380, ATRN-119, ATRN-212, AZ-20, AZZ-6738, BAY-1895344, berzosertib (M-6620, VX-970; VE-822), BKT-300, IMP-9064, M-1774, M-4344 (VX-803), M-6620, nLs-BG-129, NU-6027, RP-3500, and SC-0245.

48. The method of claim 45, wherein the CHK1 inhibitor is selected from the group consisting of AZD-7762, BEBT-260, GDC-0575, LY-2880070, PF-477736, prexasertib (ACR-368), rabusertib (LY-2603618), RG-7602, SCH-900776, SRA737, and XCCS-605B.

49. The method of claim 45, wherein the WEE1 inhibitor is selected from the group consisting of AZD1775 (MK1775), Bos-I, bosutinib, DC-859/A, Debio 0123, IMP7068, NUV-569, PD0166285, PD0407824, SC-0191, SDR-7778, SDR-7995, and ZN-c3.

50. The method of claim 45, wherein the PARG inhibitor is selected from the group consisting of PD00017273, 3-((1-Methyl-1H-pyrazol-4-yl)methyl)-N-(1-methylcyclopropyl)-1-(oxetan-3-ylmethyl)-2,4-dioxo-1,2,3,4-tetrahydroquinazoline-6-sulfonamide, and (R)—N-(1-Cyanocyclopropyl)-1-(5-(difluoromethyl)-1,3,4-thiadiazol-2-yl)-4-(3-methyl-4-(1-methylcyclopropane-1-carbonyl)piperazin-1-yl)-1H-indazole-6-sulfonamide.

51.-102. (canceled)

103. A method for identifying a subject responsive to a replication stress pathway agent (RSPA), comprising:
a) preparing a first sample and a second sample from a tumor or tumor cell acquired from the subject; wherein the first sample and the second sample are derived from different time points;
b) assessing the first sample and the second sample for a presence and/or a level of ecDNA;
c) comparing the presence and/or the level of ecDNA between the first sample and the second sample; and
d) treating the subject with the RSPA based on a differential the presence and/or the level of ecDNA of the second sample as compared to the first sample.

104.-143. (canceled)
